(19) United States
(12) Patent Application Publication
(10) Pub. No.: US 2021/0030843 A1
(43) Pub. Date: Feb. 4, 2021

(54) COMPOSITION AND METHOD FOR REDUCING CHEMOTHERAPY-INDUCED NEUTROPENIA VIA THE ADMINISTRATION OF PLINABULIN AND A G-CSF AGENT

(71) Applicant: BEYONDSPRING PHARMACEUTICALS, INC., New York, NY (US)

(72) Inventors: Ramon Mohanlal, New York, NY (US); Lan Huang, New York, NY (US); George Kenneth Lloyd, New York, NY (US)

(21) Appl. No.: 16/966,156

(22) PCT Filed: Jan. 30, 2019
(86) PCT No.: PCT/US2019/015867

§ 371 Date: Jul. 30, 2020

Related U.S. Application Data
(60) Provisional application No. 62/757,648, filed on Nov. 8, 2018.
(60) Provisional application No. 62/749,060, filed on Oct. 22, 2018.
(60) Provisional application No. 62/713,486, filed on Aug. 1, 2018.
(60) Provisional application No. 62/625,290, filed on Feb. 1, 2018.

(51) Int. Cl. A61K 38/19; A61K 31/496; A61P 37/04

(52) U.S. Cl. CPC A61K 38/193; A61K 45/06; A61P 37/04; A61K 31/496

(57) ABSTRACT

Plinabulin and one or more G-CSF drugs are used for treating a chemotherapy induced neutropenia, stimulating neutrophil survival, reducing bone pain induced by the G-CSF drug and alleviating immune suppression effect induced by the G-CSF drug. For example, docetaxel-induced neutropenia can be reduced by-co-administering plinabulin and one or more G-CSF compounds.

### CROSS-REFERENCE TO RELATED APPLICATIONS

This application is a 371 of international PCT Application No. PCT/US2019/015867, filed on Jan. 30, 2019, which claims benefit of U.S. Provisional Application No. 62/757,648, filed on Nov. 8, 2018, and U.S. Provisional Application No. 62/749,060, filed Oct. 22, 2018, and U.S. Provisional Application No. 62/713,486, filed on Aug. 1, 2018, and U.S. Provisional Application No. 62/625,290, filed on Feb. 1, 2018, the disclosures of which are incorporated herein by reference in their entireties.

### BACKGROUND

### Field

The present invention relates to the field of chemistry and medicine. More particularly, the present invention relates to method of reducing or ameliorating neutropenia using Plinabulin.

### Description of the Related Art

Myelosuppression is the primary toxicity of many chemotherapy regimens which often limits applicability. Both the duration of Grade 4 neutropenia and the depth of the neutrophil nadir have been correlated to severe and life-threatening infections. As a result, the prevention of neutropenia is a major goal for oncology practitioners for both safety and cost-efficiency and quality of life.

Neutropenia is a frequent and potentially life-threatening complication of cytotoxic myelosuppressive chemotherapy. Research has shown that patients who develop neutropenia are more susceptible to infections which often required treatment with antibiotics and in severe cases require hospitalization. Moreover, severe neutropenia often necessitates modification of the chemotherapy regimen, thereby compromising the ultimate success of the anticancer treatment plan.

### SUMMARY

Some embodiments relate to a method of treating a chemotherapy induced neutropenia, comprising co-administering plinabulin and one or more G-CSF compounds.

Some embodiments relate to a method of reducing bone pain, comprising administering an effective amount of plinabulin. In some embodiments, the bone pain is induced by G-CSF drug. In some embodiments, the bone pain is induced by pegfilgrastim.

Some embodiments relate to a method of stimulating neutrophil survival, comprising co-administering plinabulin and one or more G-CSF compounds.

Some embodiments relate to a method of treating a patient being administered with a docetaxel in an amount sufficient to cause neutropenia, the method comprising co-administering plinabulin and one or more G-CSF compounds to alleviate or prevent neutrophil reduction in the patient.

Some embodiments relate to a method of treating docetaxel induced neutropenia in a subject, comprising co-administering plinabulin and one or more G-CSF compounds, wherein plinabulin is administered at a dose in the range of about 1 mg/m² to about 50 mg/m².

Some embodiments relate to a method of treating docetaxel induced neutropenia in a subject having advanced breast cancer (e.g., early or metastatic breast cancer), comprising: identifying a patient having advanced or metastatic breast cancer; and co-administering plinabulin and one or more G-CSF compounds, wherein plinabulin is administered at a dose in the range of about 1 mg/m² to about 50 mg/m².

Some embodiments relate to a method of treating docetaxel induced neutropenia in a subject having non-small cell lung cancer, comprising identifying a patient having non-small cell lung cancer; and co-administering plinabulin and one or more G-CSF compounds, wherein plinabulin is administered at a dose in the range of about 1 mg/m² to about 50 mg/m².

Some embodiments relate to a method of treating docetaxel induced neutropenia in a subject having hormone refractory metastatic prostate cancer, comprising: identifying a patient having hormone refractory metastatic prostate cancer; and co-administering plinabulin and one or more G-CSF compounds, wherein plinabulin is administered at a dose in the range of about 1 mg/m² to about 50 mg/m².

Some embodiments relate to a method of stimulating neutrophil survival, comprising co-administering plinabulin and one or more G-CSF compounds, wherein plinabulin is administered at a dose in the range of about 1 mg/m² to about 50 mg/m².

Some embodiments relate to a pharmaceutical composition comprising about 1 mg to about 150 mg, 1 mg to about 100 mg or about 1 mg to about 40 mg of plinabulin.

Some embodiments relate to a sterile container comprising a docetaxel, and about 1 mg to about 150 mg, 1 mg to about 100 mg or about 1 mg to about 40 mg of plinabulin, wherein the docetaxel and the plinabulin are provided in two separate sterile containers.

### BRIEF DESCRIPTION OF THE DRAWINGS

FIG. 1 is a graph showing the change of neutrophil count through time with the treatment of plinabulin versus pegfligrastim.

FIG. 2 is a graph measuring the change of median average neutrophil count (ANC) during the first 10 days of cycle 1 chemotherapy treatment for patients receiving the plinabulin and G-CSF combination or just G-CSF drug.

FIG. 3 is a bar graph showing the incidence of grade 3/4 neutropenia in cycle 1 of TAC treatment for breast cancer patients who received the plinabulin and G-CSF combination or just G-CSF.

FIG. 4 is a bar graph showing the incidence of bone pain in cycle 1 of TAC treatment for breast cancer patients who received the plinabulin and G-CSF combination or just G-CSF drug.

FIG. 5 is a graph showing the duration of bone pain in cycle 1 of TAC treatment for breast cancer patients who received the plinabulin and G-CSF combination or just G-CSF.

FIG. 6 is a graph showing the percentage of patients having a neutrophil to lymphocyte ratio (NLR) value of greater than 5 in the various treatment groups.

FIG. 7A shows the percentage of patients with NLR over 5 in plinabulin (20 mg/m2) alone, plinabulin (20 mg/m2)+neulasta 6 mg, and Neulasta (6 mg). FIG. 7B shows the percentage of patients with lymphocyte to monocyte ratio (LMR) less than 3.2 in plinabulin (20 mg/m2) alone, plinabulin (20 mg/m2)+neulasta 6 mg, and Neulasta (6 mg).

### DETAILED DESCRIPTION OF THE PREFERRED EMBODIMENT

Plinabulin, (3Z,6Z)-3-Benzylidene-6-{[5-(2-methyl-2-propanyl)-1H-imidazol-4-yl]methylene}-2,5-piperazinedione, is a synthetic analog of the natural compound phenylahistin. Plinabulin can be readily prepared according to methods and procedures detailed in U.S. Pat. Nos. 7,064,201 and 7,919,497, which are incorporated herein by reference in their entireties. In some embodiments, Plinabulin can efficiently promote antigen uptake and migration of dendritic cells to lymph nodes where tumor-specific antigens are presented by dendritic cells to prime immune effector cells. Exposure of dendritic cells to Plinabulin can induce maturation of dendritic cells and significantly increase their capacity to prime T cells. In some embodiments, Plinabulin can mediate tumor size reduction through immune modulation of the tumor microenvironment to promote anti-tumor immune enhancing effects. In some embodiments, substantial therapeutic synergies can be achieved when combining Plinabulin with G-CSF.

Plinabulin is a small molecule with tumor-inhibiting and immune-enhancing effects. Plinabulin induces dendritic cell maturation and cytokines interleukin-1β (IL-1β), IL-6, and IL-12 production, all of which are important in neutrophil survival. Plinabulin also induces production of MHCII, CD40, CD80 and CD86 and related antigen-specific T-cell activation. Plinabulin may induce maturation of dendritic cells, resulting in the release of the cytokines interleukin (IL)-1β, IL-6 and IL-12 from monocytes/dendritic cells, and the cytokines protect neutrophils against apoptosis. In particular IL-6 can be mediated in the prevention of neutrophil apoptosis and IL-1β with increased neutrophil count. Plinabulin can prevent docetaxel- or cyclophosphamide-induced neutropenia via a mechanism of action different from that of G-CSF analogues. When used for treating solid tumor, plinabulin showed protective effect against neutropenia. In a Phase 2 (Ph2) trial, the addition of Plinabulin to Docetaxel (Plin+Doc; n=38) in NSCLC patients (pts) with a measurable lesion, improved mOS with 4.6 mo vs Doc alone (n=38). DOR (a marker of immune effect) was ˜1 yr longer (P<0.05) with Plinabulin+Docetaxel vs Docetaxel alone. Plin exerted immune-enhancing effects (DOR), without increasing Immune-Related AEs (IR-AEs).

Granulocyte-colony stimulating factor (G-CSF) refers to compounds or factors that stimulate proliferation, differentiation, commitment and end cell functional activation of granulocytes in an animal, including a human subject. The term G-CSF or G-CSF variant includes all naturally occurring variants of G-CSF (with or without a leader sequence), G-CSF biosimilars, as well as G-CSF proteins derived therefrom which are modified by recombinant DNA technology, in particular fusion proteins which contain further polypeptide sequences apart from the G-CSF moiety. For example, one may: (1) increase half-life (or prepare an oral dosage form, for example) of the G-CSF molecule by, for example, decreasing the ability of proteases to act on the G-CSF molecule or adding chemical modifications to the G-CSF molecule, such as one or more polyethylene glycol molecules or enteric coatings for oral formulation which would act to change some characteristic of the G-CSF molecule as described above, such as increasing serum or other half-life or decreasing antigenicity; (2) prepare a hybrid molecule, such as combining G-CSF with part or all of another protein such as another cytokine or another protein which effects signal transduction via entry through the cell through a G-CSF-G-CSF receptor transport mechanism; or (3) increase the biological activity as in, for example, the ability to selectively stimulate neutrophils (as compared to a non-modified G-CSF molecule). G-CSF includes derivatives, mimetics, variants and chemically modified compounds or hybrids thereof as described in U.S. Pat. Nos. 5,399,345; 5,416,195; 5,981,551; 6,166,183 and 6,261,550, the contents of which are incorporated by reference in entireties. G-CSF compounds include but are not limited to filgrastim and pegfilgrastim. Examples of G-CSF include but are not limited to Neupogen® (Amgen), Tevagrastim® (Teva), Biograstim® (CT Arzneimittel), Ratiograstim® (Ratiopharm GmbH)), Zarxio® (Sandoz GmbH), Filgrastim Hexal® (Hexal AG), Neulasta® (Amgen), Granocyte® and Neutrogin® (Chugai), and Neu-up® (Kyowa Hakko), Rolontis® (Spectrum, eflapegrastim), Aiduo (mecapegfilgrastim, Hengrui), Fulphila™ (pegfilgrastim-jmdb, Mylan). G-CSF is often given to manage chemotherapy-induced severe neutropenia. G-CSF such as pegfilgrastim is a colony-stimulating factor that acts on hematopoietic cells by binding to specific cell surface receptors, thereby stimulating proliferation, differentiation, commitment, and end cell functional activation.

Febrile neutropenia (FN) is a potentially life-threatening condition characterized by the development of fever (≥38.3° C.) and docetaxel-induced neutropenia (absolute neutrophil count [ANC]<0.5×10⁹/L). The risk of severe neutropenia including FN is mitigated by reducing docetaxel dosages or extending the dosing interval of the agents. However, research has shown these measures are directly correlated to lower long-term survival rates because of the relative reduction in the dose intensity of the drug. Therefore, granulocyte colony-stimulating factor (G-CSF) such as filgrastim (Neupogen®) or pegfilgrastim (Neulasta®), can be given to manage chemotherapy-induced severe neutropenia and to allow chemotherapy to be administered more effectively. According to these guidelines, prophylactic G-CSF use is recommended for patients at significant risk of FN based on the chemotherapy regimen and patient specific risk factors. However, the prophylactic use of G-CSF has some significant limitations in terms of safety, cost and convenience of use. Treatment should be administered within 14 days of chemotherapy initiation. Moreover, G-CSF therapy cannot be initiated until 24 hours after the last dose of chemotherapy for each treatment cycle and is generally administered once per chemotherapy cycle (requires baseline complete blood count [CBC] and platelet count during therapy). The concern with administering G-CSF on the day of chemotherapy is that increasing growth of myeloid cells may increase sensitivity to cytotoxic chemotherapy agents. Since cytotoxic chemotherapy causes the most damage to rapidly growing cells, giving an agent that causes myeloid cells to grow faster while chemotherapy is present may cause more toxicity. Duration of G-CSF therapy is to attenuate chemotherapy-induced neutropenia and is dependent on the myelosuppressive potential of chemotherapy regimen employed. Patients are required to either self-administer the drug or return to the center for treatment and evaluation which is often difficult and costly for the patient.

Warnings and precautions for pegfilgrastim include splenic rupture, acute respiratory distress syndrome, allergic reactions including anaphylaxis, fatal sickle cell crisis, glomerulonephritis, capillary leak syndrome, and leukocytosis. The most common adverse reactions are bone pain and pain in an extremity which occurred in 31% and 9% of patients, respectively. Additional notable adverse events include acute febrile neutrophilic dermatosis, cutaneous vaculitis and injection site reactions.

Plinabulin can be effective in ameliorating docetaxel-related severe neutropenia (including FN) and has a better safety profile (much less bone pain) and is more convenient for the patient by reducing the number of required patient visits and potentially also reducing the burden to the healthcare system. Most importantly, plinabulin can be given after a docetaxel cycle (e.g., 30 mins or 1 hour) as opposed to 24 hours after the completion of the cycle (as prescribed by pegfilgrastim, G-CSF and its biosimilars).

Patients with solid tumors who have received plinabulin monotherapy treatment (in the absence of chemotherapy), did not experience any clinically significant deleterious changes in hematology or chemistry laboratory parameters; however, there was a significantly lower incidence of neutropenia in patients receiving plinabulin plus docetaxel compared with the docetaxel monotherapy arm.

Clinical complications of neutropenia (febrile neutropenia, infections, sepsis, and mortality) occur with Grade 4 Neutropenia, as compared to with Grade 2 or 3 Neutropenia. For regulatory approval, the FDA and Health Authorities focus on Grade 4 Neutropenia data. Grade 4 Neutropenia/Severe Neutropenia is an Absolute Neutrophil Count of <0.5×10⁹/L. In animal model studies, Plinabulin has been shown to prevent neutropenia caused by number of chemotherapies with different mechanisms: docetaxel, cisplatin, adriamycin, cyclosphosphamide, topotecan, and gemcitabine. Table 1 shows many advantages plinabulin has over G-CSF drug for treating or attenuating neutropenia.

TABLE 1
Plinabulin has a superior product profile vs. G-CSF/neulasta

| | G-CSF | Plinabulin |
|---|---|---|
| Therapy Type | Growth Factor | Anti-cancer |
| | | agent |
| Bone Pain | >20%¹ | <4% |
| (% of patients) | | |
| Hospitalization | 20% | 6% |
| (% of patients) | | |
| Dose | 24 hours after | 0.5-1 hour after |
| Administration | chemotherapy | chemotherapy |
| Therapy Type | Biologic | Small molecule |

Compared to docetaxel treatment alone, the addition of plinabulin to docetaxel significantly (p<0.0003) reduced the proportion of patients with Grade 4 neutropenia from 33.3% to 4.6% in Cycle 1. Data shows decrease in the proportions of patients with Grade 4 neutropenia (absolute neutrophil count [ANC]<0.5×10⁹/L) on Day 8, the approximate day after docetaxel administration corresponding to the largest reduction in neutrophil count. Plinabulin also reduced the clinical sequelae associated with docetaxel-induced neutropenia (sepsis, infections, hospitalizations, need for docetaxel dose reductions, and G-CSF use). Bone pain was reported in 4% of patients receiving plinabulin. Plinabulin has a favorable safety profile; the most prominent finding was Grade 3 transient hypertension in 20% and 5% of patients receiving 30 mg/m² and 20 mg/m² plinabulin, respectively.

Plinabulin can be effective for the mitigation of docetaxel-induced neutropenia. Administered by IV infusion on the same day of (approximately 30 mins or 1 hour after) docetaxel administration, plinabulin can be given in a single dose to be determined per cycle. Plinabulin has the potential to be an effective, safe (with much less bone pain), cost-effective, and convenient alternative to G-CSF for the prevention of docetaxel-induced neutropenia.

Plinabulin and G-CSF (e.g. pegfilgrastim or filgrastim) can work synergistically to treat or prevent neutropenia occurred during the chemotherapy. The chemotherapy can include treatment using chemotherapeutic agents or radiation therapy. The combination of plinabulin and G-CSF (e.g. pegfilgrastim or filgrastim) can help manage chemotherapy-induced severe neutropenia, maintain the patent's neutrophil count during treatment, and allow chemotherapy to be administered more effectively. In addition, administration of plinabulin and G-CSF drug can help shorten the duration of severe neutropenia and maintain the absolute neutrophil count within normal range.

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

“Subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate.

The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice guinea pigs, or the like.

An “effective amount” or a “therapeutically effective amount” as used herein refers to an amount of a therapeutic agent that is effective to relieve, to some extent, or to reduce the likelihood of onset of, one or more of the symptoms of a disease or condition, and includes curing a disease or condition.

“Treat,” “treatment,” or “treating,” as used herein refers to administering a compound or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from, developing, or likely developing a disease or condition.

The term “pharmaceutically acceptable salt” refers to salts that retain the biological effectiveness and properties of a compound and, which are not biologically or otherwise undesirable for use in a pharmaceutical. In many cases, the compounds disclosed herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable salts can also be formed using inorganic and organic bases. Inorganic bases from which salts can be derived include, for example, bases that contain sodium, potassium, lithium, ammonium, calcium, magnesium, iron, zinc, copper, manganese, aluminum, and the like; particularly preferred are the ammonium, potassium, sodium, calcium and magnesium salts. In some embodiments, treatment of the compounds disclosed herein with an inorganic base results in loss of a labile hydrogen from the compound to afford the salt form including an inorganic cation such as Li⁺, Na⁺, K⁺, Mg²⁺ and Ca²⁺ and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

### Method of Treatment

Plinabulin can be effective in ameliorating or treating chemotherapy related (e.g., docetaxel, TAC, or TC-related) severe neutropenia (including FN) and has a better safety profile. Patients receiving Plinabulin treatment showed less bone pain, lower hospitalization frequency, and lower frequency of grade 4 neutropenia in cycle 1 when compared with other treatment methods (e.g., G-CSF). In addition, Plinabulin treatment also resulted in minimum or less febrile neutropenia when compared with other treatment methods (e.g., G-CSF). The patient can have better quality of life due to the superior properties of Plinabulin.

For some embodiments, G-CSF can be administered with plinabulin in treating chemotherapy induced neutropenia as described herein.

In some embodiments, the chemotherapy includes only docetaxel and no other additional chemotherapeutic agent. In some embodiments, the chemotherapy does not include docetaxel.

In some embodiments, plinabulin can be co-administered with G-CSF to reduce, ameliorate, or prevent neutropenia induced by a chemotherapy or radiation therapy. In some embodiments, plinabulin can be co-administered with G-CSF to stimulate neutrophil production or proliferation. In some embodiments, plinabulin can be co-administered with G-CSF to reduce, ameliorate, or prevent neutropenia caused by docetaxel. Consistent with the benefit of neutropenia prevention, patients receiving plinabulin may require less G-CSF treatment. The co-administration of plinabulin and G-CSF can work synergistically to continuously maintain the patient's neutrophil count and reduce the risk of terminating the chemotherapy due to severe adverse effect.

Some embodiments include co-administering a composition, and/or pharmaceutical composition described herein, with an additional medicament. For example, as described above, some embodiments include co-administering plinabulin and one or more G-CSF drugs. By “co-administration,” it is meant that the two or more agents are administered in such a manner that administration of one or more agent has a broad effect at the same time as the one or more other agent, regardless of when or how they are actually administered. In one embodiment, the agents are administered simultaneously. In one such embodiment, administration in combination is accomplished by combining the agents in a single dosage form. In another embodiment, the agents are administered sequentially. In one embodiment the agents are administered through the same route, such as orally or intravenously. In another embodiment, the agents are administered through different routes, such as one being administered orally and another being administered i.v. In some embodiments, the time period between administration of one or more agent and administration of the co-administered one or more agent can be about 5 min, 10 min, 20 min, 30 min, 40 min, 45 min, 50 min, 55 min, 1 hour, 65 min, 70 min, 75 min, 90 min, 2 hours, 3 hours, 5 hours, 8 hours, 10 hours, 12 hours, 15 hours, 18 hours, 20 hours, 24 hours, 36 hours, 48 hours, 3 days, 4 days, 5 days, 6 days, 7 days, 10 days, 14 days, 21 days, 28 days, or 30 days. In some embodiments, the time period between administration of one or more agent and administration of the co-administered one or more agent can be in the range of about 1 min-5 min, 1 min-10 min, 1 min-20 min, 1 min-30 min, 1 min-40 min, 1 min-50 min, 1 min-1 h, 1 min-2 h, 1 min-4 h, 1 min-6 h, 1 min-8 h, 1 min-10 h, 1 min-12 h, 1 min-24 h, 1 min-36 h, 1 min-48 h, 1 min-60 h, 1 min-72 h, 5 min-10 min, 5 min-20 min, 5 min-30 min, 5 min-40 min, 5 min-50 min, 5 min-1 h, 5 min-75 min, 5 min-2 h, 5 min-4 h, 5 min-6 h, 5 min-8 h, 5 min-10 h, 5 min-12 h, 5 min-24 h, 5 min-36 h, 5 min-48 h, 5 min-60 h, 5 min-72 h, 10 min-20 min, 10 min-30 min, 10 min-40 min, 10 min-50 min, 10 min-1 h, 10 min-75 min, 10 min-2 h, 10 min-4 h, 10 min-6 h, 10 min-8 h, 10 min-10 h, 10 min-12 h, 10 min-24 h, 10 min-36 h, 10 min-48 h, 10 min-60 h, 10 min-72 h, 30 min-40 min, 30 min-50 min, 30 min-1 h, 30 min-75 min, 30 min-2 h, 30 min-4 h, 30 min-6 h, 30 min-8 h, 30 min-10 h, 30 min-12 h, 30 min-24 h, 30 min-36 h, 30 min-48 h, 30 min-60 h, 30 min-72 h, 1 h-2 h, 1 h-4 h, 1 h-6 h, 1 h-8 h, 1 h-10 h, 1 h-12 h, 1 h-24 h, 1 h-36 h, 1 h-48 h, 1 h-60 h, 1 h-72 h, 6 h-8 h, 6 h-10 h, 6 h-12 h, 6 h-24 h, 6 h-36 h, 6 h-48 h, 6 h-60 h, 6 h-72 h, 12 h-24 h, 12 h-36 h, 12 h-48 h, 12 h-60 h, or 12 h-72 h.

Patients receiving plinabulin and G-CSF combination treatment are less likely to require chemotherapy (e.g., docetaxel, TAC, or TC) dose reduction. The safety profile of plinabulin is better than other drugs that are used to treat or ameliorate docetaxel induced neutropenia (e.g., G-CSF treatment).

Patients receiving plinabulin and G-CSF combination treatment can show at least one of the following conditions: 1) lower incidence of Grade 4 neutropenia (absolute neutrophil count [ANC]<0.5×10⁹/L); 2) lower incidence of febrile neutropenia (FN) (ANC <0.5×10⁹/L and body temperature ≥38.3° C.); 3) higher neutrophil count during the treatment cycle; 4) lower incidence of documented infections in Cycles 1 to 4; 5) lower incidence and shorter duration of hospitalizations, and lower mortality due to FN during the treatment cycle; 6) better health-related Quality of Life; 7) shorter duration of severe neutropenia; 7) maintaining the neutrophil count within normal range and preventing the neutrophil count from overshooting above normal range. When compared with the G-CSF treatment (e.g., pegfilgrastim or filgrastim), plinabulin treatment showed lower incidence of antibiotic use, lower incidence of docetaxel dose delay, dose reduction, and/or dose discontinuation, lower Incidence, occurrence, and severity of adverse events (AEs)/serious adverse events (SAEs), lower incidence, occurrence and severity of bone pain, better systemic tolerance (physical examination and safety laboratory assessments).

In some embodiments, treating neutropenia includes reducing the likelihood or the incidence of neutropenia, reducing the duration of neutropenia, and/or maintaining the average neutrophil count of the subject to be within a range acceptable for chemotherapeutic treatment.

In some embodiments, plinabulin can be used to reduce incidence of developing bone pain. In some embodiments, plinabulin can be used to shorten the duration of bone pain. In some embodiments, the bone pain is induced by a G-CSF drug.

In some embodiments, plinabulin can be used to alleviate the immune suppression effect of G-CSF. In some embodiments, the immune suppression effect is caused by a G-CSF drug. In some embodiments, plinabulin can be used to alleviate a G-CSF drug induced immune suppression effect during a chemotherapy or radiation therapy.

In some embodiments, plinabulin can be used to reduce the NLR value (ratio between absolute neutrophil count and absolute lymphocyte count) in a patient receiving a chemotherapy. In some embodiments, plinabulin can be used to reduce the NLR value in a patient receiving a G-CSF drug during a chemotherapy. In some embodiments, the NLR value in a patient is lower than 5. In some embodiments, the method described here can reduce the incidence of the patient having a NLR value greater than 5. NLR greater than 5 is a negative predictor for poor survival. In some embodiments, the method described here can reduce the incidence of the patient having an NLR value greater than 5, and the incidence is reduced by at least 10%. In some embodiments, the method described here can reduce the incidence of the patient having a NLR value greater than 5, and the incidence is reduced by at least 10%, 20%, 30%, 40%, or 50%.

In some embodiments, plinabulin can be used to reduce the LMR value (ratio between absolute lymphocyte count and absolute monocyte count) in a patient receiving a chemotherapy. In some embodiments, the patient is also administered a G-CSF drug. LMR value less than 3.6 is a negative predictor for poor survival.

In some embodiments, the method described herein includes identifying a patient developing or at risk of developing a bone pain after receiving G-CSF treatment.

In some embodiments, the chemotherapy can independently include one or more agents selected from the group consisting of methotrexate, vinblastine, doxorubicin, cisplatin, MVAC (methotrexate, vinblastine, doxorubicin and cisplatin), docetaxel, trastuzumab, cyclophosphamide, paclitaxel, dose-dense AC followed by T (i.e., doxorubicin, cyclophosphamide, paclitaxel), TAC (docetaxel, doxorubicin, cyclophosphamide), fluorouracil, bleomycin, etoposide, vincristine, procarbazine, prednisone, BEACOPP (bleomycin, etoposide, doxorubicin, cyclophosphamide, vincristine, procarbazine, prednisone), gemcitabine, ifosfamide, carboplatin, ICE (ifosfamide, carboplatin, etoposide), rituximab, RICE (rituximab, ifosfamide, carboplatin, etoposide), CHOP-14 (cyclophosphamide, doxorubicin, vincristine, prednisone), mesna, novantrone, MINE (mesna, ifosfamide, novantrone, etoposide), dexamethasone, cytarabine DHAP (dexamethasone, cisplatin, cytarabine), methylprednisolone, ESHAP (etoposide, methylprednisolone, cisplatin, cytarabine), HyperCVAD and rituximab (cyclophosphamide, vincristine, doxorubicin, dexamethasone, rituximab), dacarbazine, vinblastine, dacarbazine-based combination (dacarbazine, cisplatin, vinblastine), dacarbazine-based combination with IL-2 and interferon alfa (dacarbazine, cisplatin, vinblastine, IL-2, interferon alfa), topotecan, MAID (mesna, doxorubicin, ifosfamine, dacarbazine), VeIP (vinblastine, ifosfamide, cisplatin), VIP (etoposide, ifosfamide, cisplatin), TIP (paclitaxel, ifosfamide, cisplatin), gemcitabine, CMF classic (cyclophosphamide, methotrexate, fluorouracil), AC (doxorubicin, cyclophosphamide), FEC (fluorouracil, epirubicin, cyclophosphamide), TC (docetaxel, cyclophosphamide), cisplatin/topotecan, paclitaxel/cisplatin, irincotecan, FOLFOX (fluorouracil, leucovorin, oxaliplatin), irincotecan/cisplatin, epirubicin/cisplatin/5-fluorouracil, epirubicin/cisplatin/capecitabine, DT-PACE (dexamethasone/thalidomide/cisplatin/doxorubicin/cyclophosphamide/etoposide), ET-PACE and bortezomib, EPOCH (etoposide, prednisone, vincristine, cyclophosphamide, doxorubicin), GDP (gemcitabine, dexamethasone, cisplatin), GDP and rituximab, FMR (fludarabine, mitoxantrone, rituximab, CHOP and rituximab (cyclophosphamide, doxorubicin, vincristine, prednisone, rituximab), cisplatin/paclitaxel, cisplatin/vinorelbine, cisplatin/docetaxel, ciaplatin/etoposide, carboplatin/paclitaxel, carboplatin/docetaxel, FOLFIRINOX (5-FU/leucovorin, irinotecan and oxaliplatin), cabazitaxel, etoposide/carboplatin, etoposide/cisplatin. In some embodiments, the chemotherapy can independently include one or more agents selected from the group consisting of methotrexate, vinblastine, doxorubicin, cisplatin, docetaxel, trastuzumab, cyclophosphamide, paclitaxel, fluorouracil, bleomycin, etoposide, vincristine, procarbazine, prednisone, gemcitabine, ifosfamide, carboplatin, mesna, novantrone, cytarabine methylprednisolone, rituximab dacarbazine, vinblastine, topotecan, gemcitabine, irincotecan, epirubicin, 5-fluorouracil, capecitabine, bortezomib, and cabazitaxel. In some embodiments, the chemotherapy does not comprises taxane. In some embodiments, the chemotherapy does not comprise docetaxel alone. In some embodiments, when the chemotherapy includes administering more than one chemotherapeutic agent, at least one of the chemotherapeutic agent is not taxane. In some embodiments, when the chemotherapy includes administering more than one chemotherapeutic agent, at least one of the chemotherapeutic agent is not docetaxel. In some embodiments, when the chemotherapy involves only one chemotherapeutic agent, the chemotherapy is not taxane. In some embodiments, when the chemotherapy involves only one chemotherapeutic agent, the chemotherapy is not docetaxel. In some embodiments, the chemotherapy comprises administering docetaxel, doxorubicin and cyclophosphamide (TAC); docetaxel and cyclophosphamide (TC); doxorubicin and cyclophosphamide (AC); docetaxel and doxorubicin (TA); docetaxel; doxorubicin; or cyclophosphamide. In some embodiments, the chemotherapy comprises administering docetaxel, doxorubicin and cyclophosphamide (TAC).

In some embodiments, the chemotherapy can include one or more agents selected from the group consisting of methotrexate, vinblastine, doxorubicin, cisplatin, MVAC (methotrexate, vinblastine, doxorubicin and cisplatin), trastuzumab, cyclophosphamide, dose-dense AC followed by T (i.e., doxorubicin, cyclophosphamide, paclitaxel), fluorouracil, bleomycin, etoposide, vincristine, procarbazine, prednisone, BEACOPP (bleomycin, etoposide, doxorubicin, cyclophosphamide, vincristine, procarbazine, prednisone), gemcitabine, ifosfamide, carboplatin, ICE (ifosfamide, carboplatin, etoposide), rituximab, RICE (rituximab, ifosfamide, carboplatin, etoposide), CHOP-14 (cyclophosphamide, doxorubicin, vincristine, prednisone), mesna, novantrone, MINE (mesna, ifosfamide, novantrone, etoposide), dexamethasone, cytarabine DHAP (dexamethasone, cisplatin, cytarabine), methylprednisolone, ESHAP (etoposide, methylprednisolone, cisplatin, cytarabine), HyperCVAD and rituximab (cyclophosphamide, vincristine, doxorubicin, dexamethasone, rituximab), dacarbazine, vinblastine, dacarbazine-based combination (dacarbazine, cisplatin, vinblastine), dacarbazine-based combination with IL-2 and interferon alfa (dacarbazine, cisplatin, vinblastine, IL-2, interferon alfa), topotecan, MAID (mesna, doxorubicin, ifosfamine, dacarbazine), VeIP (vinblastine, ifosfamide, cisplatin), VIP (etoposide, ifosfamide, cisplatin), TIP (paclitaxel, ifosfamide, cisplatin). In some embodiments, the gemcitabine, CMF classic (cyclophosphamide, methotrexate, fluorouracil), AC (doxorubicin, cyclophosphamide), FEC (fluorouracil, epirubicin, cyclophosphamide), cisplatin/topotecan, paclitaxel/cisplatin, irincotecan, FOLFOX (fluorouracil, leucovorin, oxaliplatin), irincotecan/cisplatin, epirubicin/cisplatin/5-fluorouracil, epirubicin/cisplatin/capecitabine, DT-PACE (dexamethasone/thalidomide/cisplatin/doxorubicin/cyclophosphamide/etoposide), ET-PACE and bortezomib, EPOCH (etoposide, prednisone, vincristine, cyclophosphamide, doxorubicin), GDP (gemcitabine, dexamethasone, cisplatin), GDP and rituximab, FMR (fludarabine, mitoxantrone, rituximab, CHOP and rituximab (cyclophosphamide, doxorubicin, vincristine, prednisone, rituximab), cisplatin/paclitaxel, cisplatin/vinorelbine, ciaplatin/etoposide, carboplatin/paclitaxel, FOLFIRINOX (5-FU/leucovorin, irinotecan and oxaliplatin), cabazitaxel, etoposide/carboplatin, etoposide/cisplatin. In some embodiments, the chemotherapy can include one or more agents selected from the group consisting of methotrexate, vinblastine, doxorubicin, cisplatin, trastuzumab, cyclophosphamide, fluorouracil, bleomycin, etoposide, vincristine, procarbazine, prednisone, gemcitabine, ifosfamide, carboplatin, mesna, novantrone, cytarabine methylprednisolone, rituximab dacarbazine, vinblastine, topotecan, gemcitabine, irincotecan, epirubicin, 5-fluorouracil, capecitabine, and bortezomib.

Some embodiments relate to a method of reducing or preventing neutropenia induced by chemotherapy, the method comprising co-administering plinabulin and one or more G-CSF compounds to the patient undergoing chemotherapy treatment. Some embodiments relate to a method of reducing or preventing neutropenia induced by docetaxel, the method comprising co-administering plinabulin and one or more G-CSF compounds to the patient undergoing docetaxel treatment.

Chemotherapy such as Taxotere, Adriamycin and Cyclophosphamide (TAC), and Taxotere and Cyclophosphamide (TC) can also cause severe neutropenia. TAC has a high risk (>20%) of causing FN. In some embodiments, during the TAC chemotherapy, the doxorubicin component is omitted and the TA chemotherapy is administered. For example, during the TAC treatment, in cycles 2 to 4, the doxorubicin component may be omitted at the discretion of the investigator, i.e., TC may be administered instead of TAC. Some embodiments relate to a method of reducing or preventing neutropenia induced by TAC or TC, the method comprising administering plinabulin to the patient undergoing docetaxel treatment. In some embodiments, the chemotherapy includes only TAC and no other additional chemotherapeutic agent. In some embodiments, the chemotherapy includes only TC and no other additional chemotherapeutic agent. In some embodiments, the administration schedule of TAC includes Day 1: Doxorubicin 50 mg/m2 IV, followed by cyclophosphamide 500 mg/m2 IV, followed by docetaxel 75 mg/m2 IV after a 1-hr interval. In some embodiments, the administration schedule of TC includes: Day 1: Docetaxel 75 mg/m2 IV followed by cyclophosphamide 600 mg/m2 IV.

Plinabulin is useful in preventing, treating, or ameliorating neutrophil reduction arising from chemotherapy (e.g., docetaxel, TAC, or TC) treatment.

Some embodiments relate to a method of treating a patient being administered with docetaxel in an amount sufficient to cause neutropenia, the method comprising: co-administering plinabulin and one or more G-CSF compounds to alleviate or prevent neutrophil reduction in the patient.

Some embodiments relate to a method of treating a patient being administered with chemotherapy in an amount sufficient to cause neutropenia, the method comprising: co-administering plinabulin and G-CSF to alleviate or prevent neutrophil reduction in the patient.

Some embodiments relate to co-administering plinabulin and G-CSF to relieve the degree of neutropenia and to shorten the severe duration of neutropenia.

In some embodiments, the patient has an advanced or metastatic breast cancer, early stage breast cancer, non-small cell lung cancer, refractory metastatic prostate cancer. In some embodiments, the patient has head and neck cancer, lung cancer, stomach cancer, colon cancer, pancreatic cancer, prostate cancer, breast cancer, kidney cancer, bladder cancer, ovary cancer, cervical cancer, melanoma, glioblastoma, myeloid leukemia, myeloma, lymphoma, or leukemia. In some embodiments, the patient has renal cell carcinoma, malignant melanoma, non-small cell lung cancer (NSCLC), ovarian cancer, Hodgkin's lymphoma or squamous cell carcinoma. In some embodiments, the patient has breast cancer, colon cancer, rectal cancer, lung cancer, prostate cancer, melanoma, leukemia, ovarian cancer, gastric cancer, renal cell carcinoma, liver cancer, pancreatic cancer, lymphomas and myeloma. In some embodiments, the patient has a solid tumor or hematological cancer. In some embodiments, the patient has Acute Lymphocytic Leukemia (ALL), Acute Myeloid Leukemia (AML), Adrenal Cancer, Basal and Squamous Cell Skin Cancer, Bile Duct Cancer, Bladder Cancer, Bone Cancer, Brain and Spinal Cord Tumors, Breast Cancer, Cervical Cancer, Chronic Lymphocytic Leukemia (CLL), Chronic Myeloid Leukemia (CML), Chronic Myelomonocytic Leukemia (CMML), Colorectal Cancer, Endometrial Cancer, Esophagus Cancer, eye Cancer (Ocular Melanoma and Lymphoma), Gallbladder Cancer, Gastrointestinal Carcinoid Tumors, Gastrointestinal Stromal Tumor (GIST), Gestational Trophoblastic Disease, Hodgkin Lymphoma, Kaposi Sarcoma, Kidney Cancer, Laryngeal and Hypopharyngeal Cancer, Leukemia, Liver Cancer, Lung Cancer, Lung Carcinoid Tumor, Lymphoma, Malignant Mesothelioma, Melanoma Skin Cancer, Merkel Cell Skin Cancer, Multiple Myeloma, Myelodysplastic Syndromes, Nasal Cavity and Paranasal Sinuses Cancer, Nasopharyngeal Cancer, Neuroblastoma, Non-Hodgkin Lymphoma, Non-Small Cell Lung Cancer, Oral Cavity and Oropharyngeal Cancer, Osteosarcoma, Ovarian Cancer, Pancreatic Cancer, Prostate Cancer, Retinoblastoma, Rhabdomyosarcoma, Salivary Gland Cancer, Skin Cancer, Small Cell Lung Cancer, Small Intestine Cancer, Soft Tissue Sarcoma, Stomach Cancer, Testicular Cancer, Thymus Cancer, Thyroid Cancer, Uterine Sarcoma, Vaginal Cancer, Vulvar Cancer, Waldenstrom Macroglobulinemia, or Wilms Tumor.

Some embodiments relate to treating a chemotherapy (e.g., docetaxel, TAC, or TC) induced neutropenia in a subject having advanced for metastatic breast cancer, comprising identifying a patient having advanced or metastatic breast cancer; and co-administering a pharmaceutically effective amount of plinabulin and a pharmaceutically effective amount of G-CSF compound.

Some embodiments relate to a method of treating chemotherapy (e.g., docetaxel, TAC, or TC) induced neutropenia in a subject having non-small cell lung cancer, comprising: identifying a patient having non-small cell lung cancer; and co-administering a pharmaceutically effective amount of plinabulin and a pharmaceutically effective amount of G-CSF compound.

Some embodiments relate to a method of treating chemotherapy (e.g., docetaxel, TAC, or TC) induced neutropenia in a subject having hormone refractory metastatic prostate cancer, comprising: identifying a patient having hormone refractory metastatic prostate cancer; and co-administering a pharmaceutically effective amount of plinabulin and a pharmaceutically effective amount of G-CSF compound.

In some embodiments, the neutropenia is a febrile neutropenia. In some embodiments, the neutropenia is a drug-induced neutropenia. In some embodiments, the neutropenia is a taxane-induced neutropenia.

Some embodiments relate to a method of stimulating neutrophil survival, comprising co-administering plinabulin and G-CSF, wherein the plinabulin is administered at a dose in the range of about 1 mg/m² to about 50 mg/m². Some embodiments relate to a method of stimulating neutrophil survival, comprising co-administering plinabulin and one or more G-CSF compounds.

In some embodiments, the G-CSF drug is administered using an on-body injector (e.g., Onpro® on-body Injector). In some embodiments, the G-CSF drug is administered subcutaneously.

In some embodiments, the method described herein further comprises monitoring the patient's absolute neutrophil count. In some embodiments, the method described herein further comprises monitoring the patient's absolute neutrophil count and administering G-CSF drug when the patient has an absolute neutrophil count that is lower than about 1.5×10⁹/L, about 1.0×10⁹/L, or about 0.5×10⁹/L.

In some embodiments, when plinabulin is co-administered with G-CSF to treat neutropenia, the patient has an absolute neutrophil count (ANC) of less than 500 neutrophils/mcl or an ANC of less than 1000 neutrophils/mcl and a predicted decline of less than or equal to 500 neutrophils/mcl over the following 48 hours. In some embodiments, plinabulin is co-administered with G-CSF to treat neutropenia in a patient having ANC of less than 100 neutrophils/mcl. In some embodiments, plinabulin is co-administered with G-CSF to treat neutropenia in a patient having ANC of less than 500 neutrophils/mcl. In some embodiments, plinabulin is co-administered with G-CSF to treat neutropenia in a patient having ANC of less than 1000, 900, 800, 700, 600, 500, 400, 300, 200, 100 or 50 neutrophils/mcl. In some embodiments, plinabulin is co-administered with G-CSF to treat neutropenia in a patient having ANC in the range of about 1000-100, 900-100, 800-100, 700-100, 600-100, 500-100, 400-100, 300-100, 200-100, 1000-200, 900-200, 800-200, 700-200, 600-200, 500-200, 400-200, 300-200, 1000-300, 900-300, 800-300, 700-300, 600-300, 500-300, 400-300, 1000-400, 900-400, 800-400, 700-400, 600-400, 500-400, 1000-500, 900-500, 800-500, 700-500, or 600-500 neutrophils/mcl.

In some embodiments, the plinabulin is administered at a dose in the range of about 1-50 mg/m² of the body surface area. In some embodiments, the plinabulin is administered at a dose of less than about 20 mg/m² of the body surface area. In some embodiments, the plinabulin is administered at a dose in the range of about 10-30 or about 15-25 mg/m² of the body surface area. In some embodiments, the plinabulin is administered at a dose in the range of about 1-2, 1-3, 1-4, 1-5, 1-6, 1-7, 1-8, 1-9, 1-10, 1-11, 1-12, 1-13, 1-13.75, 1-14, 1-15, 1-16, 1-17, 1-18, 1-19, 1-20, 1-22.5, 1-25, 1-27.5, 1-30, 1.5-2, 1.5-3, 1.5-4, 1.5-5, 1.5-6, 1.5-7, 1.5-8, 1.5-9, 1.5-10, 1.5-11, 1.5-12, 1.5-13, 1.5-13.75, 1.5-14, 1.5-15, 1.5-16, 1.5-17, 1.5-18, 1.5-19, 1.5-20, 1.5-22.5, 1.5-25, 1.5-27.5, 1.5-30, 2.5-2, 2.5-3, 2.5-4, 2.5-5, 2.5-6, 2.5-7, 2.5-8, 2.5-9, 2.5-10, 2.5-11, 2.5-12, 2.5-13, 2.5-13.75, 2.5-14, 2.5-15, 2.5-16, 2.5-17, 2.5-18, 2.5-19, 2.5-20, 2.5-22.5, 2.5-25, 2.5-27.5, 2.5-30, 2.5-7.5, 3-4, 3-5, 3-6, 3-7, 3-8, 3-9, 3-10, 3-11, 3-12, 3-13, 3-13.75, 3-14, 3-15, 3-16, 3-17, 3-18, 3-19, 3-20, 3-22.5, 3-25, 3-27.5, 3-30, 3.5-6.5, 3.5-13.75, 3.5-15, 2.5-17.5, 4-5, 4-6, 4-7, 4-8, 4-9, 4-10, 4-11, 4-12, 4-13, 4-13.75, 4-14, 4-15, 4-16, 4-17, 4-18, 4-19, 4-20, 4-22.5, 4-25, 4-27.5, 4-30, 5-6, 5-7, 5-8, 5-9, 5-10, 5-11, 5-12, 5-13, 5-13.75, 5-14, 5-15, 5-16, 5-17, 5-18, 5-19, 5-20, 5-22.5, 5-25, 5-27.5, 5-30, 6-7, 6-8, 6-9, 6-10, 6-11, 6-12, 6-13, 6-13.75, 6-14, 6-15, 6-16, 6-17, 6-18, 6-19, 6-20, 6-22.5, 6-25, 6-27.5, 6-30, 7-8, 7-9, 7-10, 7-11, 7-12, 7-13, 7-13.75, 7-14, 7-15, 7-16, 7-17, 7-18, 7-19, 7-20, 7-22.5, 7-25, 7-27.5, 7-30, 7.5-12.5, 7.5-13.5, 7.5-15, 8-9, 8-10, 8-11, 8-12, 8-13, 8-13.75, 8-14, 8-15, 8-16, 8-17, 8-18, 8-19, 8-20, 8-22.5, 8-25, 8-27.5, 8-30, 9-10, 9-11, 9-12, 9-13, 9-13.75, 9-14, 9-15, 9-16, 9-17, 9-18, 9-19, 9-20, 9-22.5, 9-25, 9-27.5, 9-30, 10-11, 10-12, 10-13, 10-13.75, 10-14, 10-15, 10-16, 10-17, 10-18, 10-19, 10-20, 10-22.5, 10-25, 10-27.5, 10-30, 11.5-15.5, 12.5-14.5, 7.5-22.5, 8.5-32.5, 9.5-15.5, 15.5-24.5, 5-35, 17.5-22.5, 22.5-32.5, 25-35, 25.5-24.5, 27.5-32.5, 2-20, t 2.5-22.5, or 9.5-21.5 mg/m², of the body surface area. In some embodiments, the plinabulin is administered at a dose of about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40 mg/m² of the body surface area. In some embodiments, the plinabulin is administered at a dose less than about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40 mg/m² of the body surface area. In some embodiments, the plinabulin is administered at a dose greater than about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40, 41, 42, 43, 44, 45, 46, 47, 48, 49, 50 mg/m² of the body surface area.

In some embodiments, the plinabulin is administered at a single dose per treatment cycle. In some embodiments, the plinabulin is administered at two or more doses per treatment cycle. In some embodiments, the treatment cycle is a 21-day treatment cycle.

In some embodiments, the total dosage of plinabulin administered in a 21-day cycle is in the range of about 1-50 mg/m² of the body surface area. In some embodiments, the total dosage of plinabulin administered in a 21-day cycle is less than about 20 mg/m² of the body surface area. In some embodiments, the total dosage of plinabulin administered in a 21-day cycle is in the range of about 10-30 or about 15-25 mg/m² of the body surface area. In some embodiments, the total dosage of plinabulin administered in a 21-day cycle is in the range of about 1-2, 1-3, 1-4, 1-5, 1-6, 1-7, 1-8, 1-9, 1-10, 1-11, 1-12, 1-13, 1-13.75, 1-14, 1-15, 1-16, 1-17, 1-18, 1-19, 1-20, 1-22.5, 1-25, 1-27.5, 1-30, 1.5-2, 1.5-3, 1.5-4, 1.5-5, 1.5-6, 1.5-7, 1.5-8, 1.5-9, 1.5-10, 1.5-11, 1.5-12, 1.5-13, 1.5-13.75, 1.5-14, 1.5-15, 1.5-16, 1.5-17, 1.5-18, 1.5-19, 1.5-20, 1.5-22.5, 1.5-25, 1.5-27.5, 1.5-30, 2.5-2, 2.5-3, 2.5-4, 2.5-5, 2.5-6, 2.5-7, 2.5-8, 2.5-9, 2.5-10, 2.5-11, 2.5-12, 2.5-13, 2.5-13.75, 2.5-14, 2.5-15, 2.5-16, 2.5-17, 2.5-18, 2.5-19, 2.5-20, 2.5-22.5, 2.5-25, 2.5-27.5, 2.5-30, 2.5-7.5, 3-4, 3-5, 3-6, 3-7, 3-8, 3-9, 3-10, 3-11, 3-12, 3-13, 3-13.75, 3-14, 3-15, 3-16, 3-17, 3-18, 3-19, 3-20, 3-22.5, 3-25, 3-27.5, 3-30, 3.5-6.5, 3.5-13.75, 3.5-15, 2.5-17.5, 4-5, 4-6, 4-7, 4-8, 4-9, 4-10, 4-11, 4-12, 4-13, 4-13.75, 4-14, 4-15, 4-16, 4-17, 4-18, 4-19, 4-20, 4-22.5, 4-25, 4-27.5, 4-30, 5-6, 5-7, 5-8, 5-9, 5-10, 5-11, 5-12, 5-13, 5-13.75, 5-14, 5-15, 5-16, 5-17, 5-18, 5-19, 5-20, 5-22.5, 5-25, 5-27.5, 5-30, 6-7, 6-8, 6-9, 6-10, 6-11, 6-12, 6-13, 6-13.75, 6-14, 6-15, 6-16, 6-17, 6-18, 6-19, 6-20, 6-22.5, 6-25, 6-27.5, 6-30, 7-8, 7-9, 7-10, 7-11, 7-12, 7-13, 7-13.75, 7-14, 7-15, 7-16, 7-17, 7-18, 7-19, 7-20, 7-22.5, 7-25, 7-27.5, 7-30, 7.5-12.5, 7.5-13.5, 7.5-15, 8-9, 8-10, 8-11, 8-12, 8-13, 8-13.75, 8-14, 8-15, 8-16, 8-17, 8-18, 8-19, 8-20, 8-22.5, 8-25, 8-27.5, 8-30, 9-10, 9-11, 9-12, 9-13, 9-13.75, 9-14, 9-15, 9-16, 9-17, 9-18, 9-19, 9-20, 9-22.5, 9-25, 9-27.5, 9-30, 10-11, 10-12, 10-13, 10-13.75, 10-14, 10-15, 10-16, 10-17, 10-18, 10-19, 10-20, 10-22.5, 10-25, 10-27.5, 10-30, 11.5-15.5, 12.5-14.5, 7.5-22.5, 8.5-32.5, 9.5-15.5, 15.5-24.5, 5-35, 17.5-22.5, 22.5-32.5, 25-35, 25.5-24.5, 27.5-32.5, 2-20, t 2.5-22.5, or 9.5-21.5 mg/m², of the body surface area. In some embodiments, the total dosage of plinabulin administered in a 21-day cycle is about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40 mg/m² of the body surface area. In some embodiments, the total dosage of plinabulin administered in a 21-day cycle is less than about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40 mg/m² of the body surface area. In some embodiments, the total dosage of plinabulin administered in a 21-day cycle is greater than about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40, 41, 42, 43, 44, 45, 46, 47, 48, 49, 50 mg/m² of the body surface area.

In some embodiments, when a single dose of plinabulin is administered once per chemotherapy (e.g., docetaxel, TAC, or TC) treatment cycle (e.g., 21 day), the total amount of plinabulin administered per treatment cycle of the chemotherapy is in the range of about 1-2, 1-3, 1-4, 1-5, 1-6, 1-7, 1-8, 1-9, 1-10, 1-11, 1-12, 1-13, 1-13.75, 1-14, 1-15, 1-16, 1-17, 1-18, 1-19, 1-20, 1-22.5, 1-25, 1-27.5, 1-30, 1.5-2, 1.5-3, 1.5-4, 1.5-5, 1.5-6, 1.5-7, 1.5-8, 1.5-9, 1.5-10, 1.5-11, 1.5-12, 1.5-13, 1.5-13.75, 1.5-14, 1.5-15, 1.5-16, 1.5-17, 1.5-18, 1.5-19, 1.5-20, 1.5-22.5, 1.5-25, 1.5-27.5, 1.5-30, 2.5-2, 2.5-3, 2.5-4, 2.5-5, 2.5-6, 2.5-7, 2.5-8, 2.5-9, 2.5-10, 2.5-11, 2.5-12, 2.5-13, 2.5-13.75, 2.5-14, 2.5-15, 2.5-16, 2.5-17, 2.5-18, 2.5-19, 2.5-20, 2.5-22.5, 2.5-25, 2.5-27.5, 2.5-30, 2.5-7.5, 3-4, 3-5, 3-6, 3-7, 3-8, 3-9, 3-10, 3-11, 3-12, 3-13, 3-13.75, 3-14, 3-15, 3-16, 3-17, 3-18, 3-19, 3-20, 3-22.5, 3-25, 3-27.5, 3-30, 3.5-6.5, 3.5-13.75, 3.5-15, 2.5-17.5, 4-5, 4-6, 4-7, 4-8, 4-9, 4-10, 4-11, 4-12, 4-13, 4-13.75, 4-14, 4-15, 4-16, 4-17, 4-18, 4-19, 4-20, 4-22.5, 4-25, 4-27.5, 4-30, 5-6, 5-7, 5-8, 5-9, 5-10, 5-11, 5-12, 5-13, 5-13.75, 5-14, 5-15, 5-16, 5-17, 5-18, 5-19, 5-20, 5-22.5, 5-25, 5-27.5, 5-30, 6-7, 6-8, 6-9, 6-10, 6-11, 6-12, 6-13, 6-13.75, 6-14, 6-15, 6-16, 6-17, 6-18, 6-19, 6-20, 6-22.5, 6-25, 6-27.5, 6-30, 7-8, 7-9, 7-10, 7-11, 7-12, 7-13, 7-13.75, 7-14, 7-15, 7-16, 7-17, 7-18, 7-19, 7-20, 7-22.5, 7-25, 7-27.5, 7-30, 7.5-12.5, 7.5-13.5, 7.5-15, 8-9, 8-10, 8-11, 8-12, 8-13, 8-13.75, 8-14, 8-15, 8-16, 8-17, 8-18, 8-19, 8-20, 8-22.5, 8-25, 8-27.5, 8-30, 9-10, 9-11, 9-12, 9-13, 9-13.75, 9-14, 9-15, 9-16, 9-17, 9-18, 9-19, 9-20, 9-22.5, 9-25, 9-27.5, 9-30, 10-11, 10-12, 10-13, 10-13.75, 10-14, 10-15, 10-16, 10-17, 10-18, 10-19, 10-20, 10-22.5, 10-25, 10-27.5, 10-30, 11.5-15.5, 12.5-14.5, 7.5-22.5, 8.5-32.5, 9.5-15.5, 15.5-24.5, 5-35, 17.5-22.5, 22.5-32.5, 25-35, 25.5-24.5, 27.5-32.5, 2-20, 2.5-22.5, or 9.5-21.5 mg/m², of the body surface area. In some embodiments, the total amount of plinabulin administered per chemotherapy treatment cycle is about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40 mg/m² of the body surface area. In some embodiments, the total amount of plinabulin administered per chemotherapy treatment cycle is less than about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40 mg/m² of the body surface area. In some embodiments, the total amount of plinabulin administered per chemotherapy treatment cycle is greater than about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40, 41, 42, 43, 44, 45, 46, 47, 48, 49, 50 mg/m² of the body surface area. In some embodiments, the total amount of plinabulin administered per chemotherapy treatment cycle is about 20 mg/m² of the body surface area.

In some embodiments, the plinabulin dose is about 5 mg-300 mg, 5 mg-200 mg, 7.5 mg-200 mg, 10 mg-100 mg, 15 mg-100 mg, 20 mg-100 mg, 30 mg-100 mg, 40 mg-100 mg, 10 mg-80 mg, 15 mg-80 mg, 20 mg-80 mg, 30 mg-80 mg, 40 mg-80 mg, 10 mg-60 mg, 15 mg-60 mg, 20 mg-60 mg, 30 mg-60 mg, about 40 mg-60 mg, 1 mg-40 mg, 1 mg-35 mg, 1 mg-30 mg, 10 mg-40 mg, 10 mg-35 mg, or 20 mg-35 mg. In some embodiments, the plinabulin administered is about 20 mg-60 mg, 27 mg-60 mg, 20 mg-45 mg, or 27 mg-45 mg. In some embodiments, the plinabulin administered is about 5 mg-7.5 mg, 5 mg-9 mg, 5 mg-10 mg, 5 mg-12 mg, 5 mg-14 mg, 5 mg-15 mg, 5 mg-16 mg, 5 mg-18 mg, 5 mg-20 mg, 5 mg-22 mg, 5 mg-24 mg, 5 mg-26 mg, 5 mg-28 mg, 5 mg-30 mg, 5 mg-32 mg, 5 mg-34 mg, 5 mg-36 mg, 5 mg-38 mg, 5 mg-40 mg, 5 mg-42 mg, 5 mg-44 mg, 5 mg-46 mg, 5 mg-48 mg, 5 mg-50 mg, 5 mg-52 mg, 5 mg-54 mg, 5 mg-56 mg, 5 mg-58 mg, 5 mg-60 mg, 7 mg-7.7 mg, 7 mg-9 mg, 7 mg-10 mg, 7 mg-12 mg, 7 mg-14 mg, 7 mg-15 mg, 7 mg-16 mg, 7 mg-18 mg, 7 mg-20 mg, 7 mg-22 mg, 7 mg-24 mg, 7 mg-26 mg, 7 mg-28 mg, 7 mg-30 mg, 7 mg-32 mg, 7 mg-34 mg, 7 mg-36 mg, 7 mg-38 mg, 7 mg-40 mg, 7 mg-42 mg, 7 mg-44 mg, 7 mg-46 mg, 7 mg-48 mg, 7 mg-50 mg, 7 mg-52 mg, 7 mg-54 mg, 7 mg-56 mg, 7 mg-58 mg, 7 mg-60 mg, 9 mg-10 mg, 9 mg-12 mg, 9 mg-14 mg, 9 mg-15 mg, 9 mg-16 mg, 9 mg-18 mg, 9 mg-20 mg, 9 mg-22 mg, 9 mg-24 mg, 9 mg-26 mg, 9 mg-28 mg, 9 mg-30 mg, 9 mg-32 mg, 9 mg-34 mg, 9 mg-36 mg, 9 mg-38 mg, 9 mg-40 mg, 9 mg-42 mg, 9 mg-44 mg, 9 mg-46 mg, 9 mg-48 mg, 9 mg-50 mg, 9 mg-52 mg, 9 mg-54 mg, 9 mg-56 mg, 9 mg-58 mg, 9 mg-60 mg, 10 mg-12 mg, 10 mg-14 mg, 10 mg-15 mg, 10 mg-16 mg, 10 mg-18 mg, 10 mg-20 mg, 10 mg-22 mg, 10 mg-24 mg, 10 mg-26 mg, 10 mg-28 mg, 10 mg-30 mg, 10 mg-32 mg, 10 mg-34 mg, 10 mg-36 mg, 10 mg-38 mg, 10 mg-40 mg, 10 mg-42 mg, 10 mg-44 mg, 10 mg-46 mg, 10 mg-48 mg, 10 mg-50 mg, 10 mg-52 mg, 10 mg-54 mg, 10 mg-56 mg, 10 mg-58 mg, 10 mg-60 mg, 12 mg-14 mg, 12 mg-15 mg, 12 mg-16 mg, 12 mg-18 mg, 12 mg-20 mg, 12 mg-22 mg, 12 mg-24 mg, 12 mg-26 mg, 12 mg-28 mg, 12 mg-30 mg, 12 mg-32 mg, 12 mg-34 mg, 12 mg-36 mg, 12 mg-38 mg, 12 mg-40 mg, 12 mg-42 mg, 12 mg-44 mg, 12 mg-46 mg, 12 mg-48 mg, 12 mg-50 mg, 12 mg-52 mg, 12 mg-54 mg, 12 mg-56 mg, 12 mg-58 mg, 12 mg-60 mg, 15 mg-16 mg, 15 mg-18 mg, 15 mg-20 mg, 15 mg-22 mg, 15 mg-24 mg, 15 mg-26 mg, 15 mg-28 mg, 15 mg-30 mg, 15 mg-32 mg, 15 mg-34 mg, 15 mg-36 mg, 15 mg-38 mg, 15 mg-40 mg, 15 mg-42 mg, 15 mg-44 mg, 15 mg-46 mg, 15 mg-48 mg, 15 mg-50 mg, 15 mg-52 mg, 15 mg-54 mg, 15 mg-56 mg, 15 mg-58 mg, 15 mg-60 mg, 17 mg-18 mg, 17 mg-20 mg, 17 mg-22 mg, 17 mg-24 mg, 17 mg-26 mg, 17 mg-28 mg, 17 mg-30 mg, 17 mg-32 mg, 17 mg-34 mg, 17 mg-36 mg, 17 mg-38 mg, 17 mg-40 mg, 17 mg-42 mg, 17 mg-44 mg, 17 mg-46 mg, 17 mg-48 mg, 17 mg-50 mg, 17 mg-52 mg, 17 mg-54 mg, 17 mg-56 mg, 17 mg-58 mg, 17 mg-60 mg, 20 mg-22 mg, 20 mg-24 mg, 20 mg-26 mg, 20 mg-28 mg, 20 mg-30 mg, 20 mg-32 mg, 20 mg-34 mg, 20 mg-36 mg, 20 mg-38 mg, 20 mg-40 mg, 20 mg-42 mg, 20 mg-44 mg, 20 mg-46 mg, 20 mg-48 mg, 20 mg-50 mg, 20 mg-52 mg, 20 mg-54 mg, 20 mg-56 mg, 20 mg-58 mg, 20 mg-60 mg, 22 mg-24 mg, 22 mg-26 mg, 22 mg-28 mg, 22 mg-30 mg, 22 mg-32 mg, 22 mg-34 mg, 22 mg-36 mg, 22 mg-38 mg, 22 mg-40 mg, 22 mg-42 mg, 22 mg-44 mg, 22 mg-46 mg, 22 mg-48 mg, 22 mg-50 mg, 22 mg-52 mg, 22 mg-54 mg, 22 mg-56 mg, 22 mg-58 mg, 22 mg-60 mg, 25 mg-26 mg, 25 mg-28 mg, 25 mg-30 mg, 25 mg-32 mg, 25 mg-34 mg, 25 mg-36 mg, 25 mg-38 mg, 25 mg-40 mg, 25 mg-42 mg, 25 mg-44 mg, 25 mg-46 mg, 25 mg-48 mg, 25 mg-50 mg, 25 mg-52 mg, 25 mg-54 mg, 25 mg-56 mg, 25 mg-58 mg, 25 mg-60 mg, 27 mg-28 mg, 27 mg-30 mg, 27 mg-32 mg, 27 mg-34 mg, 27 mg-36 mg, 27 mg-38 mg, 27 mg-40 mg, 27 mg-42 mg, 27 mg-44 mg, 27 mg-46 mg, 27 mg-48 mg, 27 mg-50 mg, 27 mg-52 mg, 27 mg-54 mg, 27 mg-56 mg, 27 mg-58 mg, 27 mg-60 mg, 30 mg-32 mg, 30 mg-34 mg, 30 mg-36 mg, 30 mg-38 mg, 30 mg-40 mg, 30 mg-42 mg, 30 mg-44 mg, 30 mg-46 mg, 30 mg-48 mg, 30 mg-50 mg, 30 mg-52 mg, 30 mg-54 mg, 30 mg-56 mg, 30 mg-58 mg, 30 mg-60 mg, 33 mg-34 mg, 33 mg-36 mg, 33 mg-38 mg, 33 mg-40 mg, 33 mg-42 mg, 33 mg-44 mg, 33 mg-46 mg, 33 mg-48 mg, 33 mg-50 mg, 33 mg-52 mg, 33 mg-54 mg, 33 mg-56 mg, 33 mg-58 mg, 33 mg-60 mg, 36 mg-38 mg, 36 mg-40 mg, 36 mg-42 mg, 36 mg-44 mg, 36 mg-46 mg, 36 mg-48 mg, 36 mg-50 mg, 36 mg-52 mg, 36 mg-54 mg, 36 mg-56 mg, 36 mg-58 mg, 36 mg-60 mg, 40 mg-42 mg, 40 mg-44 mg, 40 mg-46 mg, 40 mg-48 mg, 40 mg-50 mg, 40 mg-52 mg, 40 mg-54 mg, 40 mg-56 mg, 40 mg-58 mg, 40 mg-60 mg, 43 mg-46 mg, 43 mg-48 mg, 43 mg-50 mg, 43 mg-52 mg, 43 mg-54 mg, 43 mg-56 mg, 43 mg-58 mg, 42 mg-60 mg, 45 mg-48 mg, 45 mg-50 mg, 45 mg-52 mg, 45 mg-54 mg, 45 mg-56 mg, 45 mg-58 mg, 45 mg-60 mg, 48 mg-50 mg, 48 mg-52 mg, 48 mg-54 mg, 48 mg-56 mg, 48 mg-58 mg, 48 mg-60 mg, 50 mg-52 mg, 50 mg-54 mg, 50 mg-56 mg, 50 mg-58 mg, 50 mg-60 mg, 52 mg-54 mg, 52 mg-56 mg, 52 mg-58 mg, or 52 mg-60 mg. In some embodiments, the plinabulin dose is greater than about 5 mg, about 10 mg, about 12.5 mg, about 13.5 mg, about 15 mg, about 17.5 mg, about 20 mg, about 22.5 mg, about 25 mg, about 27 mg, about 30 mg, about 35 mg, about 40 mg, about 50 mg, about 60 mg, about 70 mg, about 80 mg, about 90 mg, about 100 mg, about 125 mg, about 150 mg, or about 200 mg. In some embodiments, the plinabulin dose is about less than about 5 mg, about 10 mg, about 12.5 mg, about 13.5 mg, about 15 mg, about 17.5 mg, about 20 mg, about 22.5 mg, about 25 mg, about 27 mg, about 30 mg, about 35 mg, about 40 mg, about 50 mg, about 60 mg, about 70 mg, about 80 mg, about 90 mg, about 100 mg, about 125 mg, about 150 mg, or about 200 mg.

In some embodiments, the neutropenia is induced by a chemotherapy. The administration period can be a multi-week treatment cycle as long as the tumor remains under control and the regimen is clinically tolerated. In some embodiments, the chemotherapy and plinabulin can be administered once every three weeks. In some embodiments, the chemotherapy and plinabulin can be administered once every week, once every two weeks, once every three weeks, once every four weeks, once evert five weeks, or once every six weeks. In some embodiments, the chemotherapy and Plinabulin can be administered once a week, and preferably once on each of day 1 and day 8 of a three-week (21 day) treatment cycle. In some embodiments, the chemotherapy and Plinabulin can be administered once a week, twice a week, three times per week, four times per week, five times per week, six times per week, or daily during a one-week, two-week, three-week, four-week, or five-week treatment cycle. The administration can be on the same or different day of each week in the treatment cycle. In some embodiments, the plinabulin is administered prior to the chemotherapy administration. In some embodiments, the plinabulin is administered concurrently with the chemotherapy administration. In some embodiments, the plinabulin is administered after the chemotherapy administration.

The G-CSF drug described herein includes both short acting or long acting G-CSF drugs. In some embodiments, the long acting G-CSF can include both drugs having pegylated linker or other linkers to achieve extended release effect. In some embodiments, the G-CSF drug is a short acting drug that requires daily administration for up to two weeks or until the ANC has reached a level acceptable for a patient undergoing chemotherapy. In some embodiments, the G-CSF drug is a long acting drug that does not require daily administration. In some embodiments, the G-CSF drug is a long acting drug that requires one dose per treatment cycle. In some embodiments, the long acting G-CSF drug can be pegfilgrastim, eflapegrastim, macafilgrastim. In some embodiments, the short acting G-CSF can be filgrastim. In some embodiments, the G-CSF drug can be selected from Neupogen® (Amgen), Tevagrastim® (Teva), Biograstim® (CT Arzneimittel), Ratiograstim® (Ratiopharm GmbH)), Zarxio® (Sandoz GmbH), Filgrastim Hexal® (Hexal AG), Neulasta® (Amgen), Granocyte® and Neutrogin® (Chugai), and Neu-up® (Kyowa Hakko), Rolontis® (Spectrum, eflapegrastim), Aiduo (mecapegfilgrastim, Hengrui), and Fulphila™ (pegfilgrastim-jmdb, Mylan).

In some embodiments, during the chemotherapy treatment cycle, the chemotherapeutic agent(s) is only administered once at the beginning of the treatment cycle, followed by the co-administration of plinabulin and G-CSF once, twice, three times, four times, five times, or six times during the treatment cycle. In some embodiments, during the chemotherapy treatment cycle, the chemotherapeutic agent(s) is only administered once at the beginning of the treatment cycle, followed by the co-administration of plinabulin and G-CSF once every week, once every two weeks, once every three weeks, once every four weeks, once evert five weeks, or once every six weeks. In some embodiments, during the chemotherapy treatment cycle, the chemotherapeutic agent(s) is only administered once at the beginning of the treatment cycle, followed by co-administration of plinabulin and G-CSF once a week, twice a week, three times per week, four times per week, five times per week, six times per week, or daily during a one-week, two-week, three-week, four-week, or five-week treatment cycle. In some embodiments, during the chemotherapy treatment cycle, the chemotherapeutic agent(s) is only administered once at the beginning of the treatment cycle, followed by administration of plinabulin once per treatment cycle, and then followed by administration of G-CSF, wherein the G-CSF drug can be either administered once per treatment cycle when a long acting G-CSF is used or multiple times per treatment cycle when a short acting G-CSF is used. The administration of the G-CSF drug is performed ≥24 hours after the administration of the chemotherapy. In some embodiments, during the chemotherapy treatment cycle, the chemotherapeutic agent(s) is administered one day 1 of the treatment cycle, followed by administration of plinabulin once on day 1 per treatment cycle, and then followed by administration of G-CSF on day 2 per treatment cycle. In some embodiments, when the chemotherapy treatment involves more than one chemotherapeutic agents, the chemotherapeutic agents are administered on day 1 and 2 of the treatment cycle, plinabulin is administered on day 1 after the first chemotherapeutic agent is administered, and then followed by administration of G-CSF on day 3 per treatment cycle. In some embodiments, when the chemotherapy treatment involves more than one chemotherapeutic agents, the chemotherapeutic agents are administered on day 1, 2, and 3 of the treatment cycle, plinabulin is administered on day 1 after the first chemotherapeutic agent is administered, and then followed by administration of G-CSF on day 4 per treatment cycle. In some embodiments, the G-CSF drug can be either administered once per treatment cycle when a long acting G-CSF is used or multiple times per treatment cycle when a short acting G-CSF is used. The administration of the G-CSF drug is performed ≥24 hours after the administration of the chemotherapy.

In some embodiments, the neutropenia is induced by a docetaxel. The administration period can be a multi-week treatment cycle as long as the tumor remains under control and the regimen is clinically tolerated. In some embodiments, docetaxel and plinabulin can be administered once every three weeks. In some embodiments, docetaxel and plinabulin can be administered once every week, once every two weeks, once every three weeks, once every four weeks, once evert five weeks, or once every six weeks. In some embodiments, docetaxel and Plinabulin can be administered once a week, and preferably once on each of day 1 and day 8 of a three-week (21 day) treatment cycle. In some embodiments, docetaxel and Plinabulin can be administered once a week, twice a week, three times per week, four times per week, five times per week, six times per week, or daily during a one-week, two-week, three-week, four-week, or five-week treatment cycle. The administration can be on the same or different day of each week in the treatment cycle. In some embodiments, the plinabulin is administered prior to the docetaxel administration. In some embodiments, the plinabulin is administered concurrently with the docetaxel administration. In some embodiments, the plinabulin is administered after the docetaxel administration.

In some embodiments, plinabulin and G-CSF is co-administered after the chemotherapy administration. When plinabulin and/or G-CSF is administered after the administration of a chemotherapy, it refers to administering plinabulin and/or G-CSF after the last chemotherapeutic agent(s) of the chemotherapy has been completely administered to the patients. For example, administering plinabulin about 30 mins after the administration of a TAC chemotherapy refers to begin the plinabulin administration about 30 mins after the administration of the last chemotherapeutic agent (e.g., docetaxel) has been completed. In some embodiments, the plinabulin is administered about 1 min, 5 min, 10 min, 15 min, 20 min, 25 min, 30 min, 45 min, 50 min, 1 h, 75 min, 1.5 h, 2 h, 2.5 h, 3 h, 4 h, 5 h, 6 h, 7 h, 8 h, 9 h, 10 h, 11 h, or 12 h after the administration of the chemotherapy. In some embodiments, the plinabulin is administered in less than about 1 min, 5 min, 10 min, 15 min, 20 min, 25 min, 30 min, 45 min, 50 min, 1 h, 75 min, 1.5 h, 2 h, 2.5 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, or 24 h after the administration of the chemotherapy. In some embodiments, the plinabulin is administered in more than about 1 min, 5 min, 10 min, 15 min, 20 min, 25 min, 30 min, 45 min, 50 min, 1 h, 75 min, 1.5 h, 2 h, 2.5 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, or 24 h after the administration of the chemotherapy. In some embodiments, the plinabulin is administered in about 1 min-5 min, 1 min-10 min, 1 min-15 min, 1 min-20 min, 1 min-25 min, 1 min-30 min, 1 min-45 min, 1 min-1 h, 1 min-75 min, 1 min-90 min, 1 min-120 min, 0.25 h-0.5 h, 0.25-0.75 h, 15 min-45 min, 15 min-75 min, 15 min-90 min, 15 min-120 min, 0.25-1 h, 30 min-45 min, 30 min-75 min, 30 min-90 min, 0.5 h-1 h, 0.5 h-2 h, 0.5 h-2.5 h, 1 h-2 h, 1 h-3 h, 1 h-5 h after the administration of the chemotherapy. In some embodiments, plinabulin is administered 30 mins after the chemotherapy administration. In some embodiments, plinabulin is administered in less than 1 hour after the chemotherapy administration.

In some embodiments, plinabulin and G-CSF can be administered after the chemotherapy administration. When plinabulin and/or G-CSF is administered after the administration of a chemotherapy, it refers to administering plinabulin and/or G-CSF after the last chemotherapeutic agent(s) of the chemotherapy has been completely administered to the patients. For example, administering plinabulin about 30 mins after the administration of a TAC chemotherapy refers to begin the plinabulin administration about 30 mins after the administration of the last chemotherapeutic agent (e.g., docetaxel) has been completed. In some embodiments, the plinabulin is administered about 1 min, 5 min, 10 min, 15 min, 20 min, 25 min, 30 min, 45 min, 50 min, 1 h, 75 min, 1.5 h, 2 h, 2.5 h, 3 h, 4 h, 5 h, 6 h, 7 h, 8 h, 9 h, 10 h, 11 h, or 12 h after the administration of the chemotherapy. In some embodiments, the plinabulin is administered in less than about 1 min, 5 min, 10 min, 15 min, 20 min, 25 min, 30 min, 45 min, 50 min, 1 h, 75 min, 1.5 h, 2 h, 2.5 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, or 24 h after the administration of the chemotherapy. In some embodiments, the plinabulin is administered in more than about 1 min, 5 min, 10 min, 15 min, 20 min, 25 min, 30 min, 45 min, 50 min, 1 h, 75 min, 1.5 h, 2 h, 2.5 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, or 24 h after the administration of the chemotherapy. In some embodiments, the plinabulin is administered in about 1 min-5 min, 1 min-10 min, 1 min-15 min, 1 min-20 min, 1 min-25 min, 1 min-30 min, 1 min-45 min, 1 min-1 h, 1 min-75 min, 1 min-90 min, 1 min-120 min, 0.25 h-0.5 h, 0.25-0.75 h, 15 min-45 min, 10 min-20 min, 10-30 min, 10-40 min, 10-50 min, 15 min-75 min, 15 min-90 min, 15 min-120 min, 20 min-30 min, 20 min-40 min, 20 min-50 min, 25 min-35 min, 28 min-32 min, 0.25-1 h, 30 min-45 min, 30 min-75 min, 30 min-90 min, 0.5 h-1 h, 0.5 h-2 h, 0.5 h-2.5 h, 1 h-2 h, 1 h-3 h, 1 h-5 h after the administration of the chemotherapy. In some embodiments, plinabulin is administered about 30 mins after the chemotherapy administration. In some embodiments, plinabulin is administered in less than 1 hour after the chemotherapy administration.

In some embodiments, G-CSF is administered after the chemotherapy administration. In some embodiments, G-CSF (if short acting G-CSF is used, then the first dose of G-CSF) is administered about 5 h, 10 h, 12 h, 15 h, 20 h, 24 h, 30 h, 36 h, 41 h, 48 h, or 54 h after the administration of the chemotherapy. In some embodiments, G-CSF (if short acting G-CSF is used, then the first dose of G-CSF) is administered in less than about 24 h, 30 h, 36 h, 42 h, 48 h, 54 h, or 60 h after the administration of the chemotherapy. In some embodiments, G-CSF (if short acting G-CSF is used, then the first dose of G-CSF) is administered in more than about 10 h, 12 h, 15 h, 20 h, 24 h, 30 h, 36 h, 41 h, 48 h, or 54 h after the administration of the chemotherapy. In some embodiments, G-CSF (if short acting G-CSF is used, then the first dose of G-CSF) is administered in about 10 h-36 h, 10 h-48 h, 10 h-54 h, 24 h-36 h, 24 h-48 h, 24 h-54 h, or 24 h-60 h after the administration of the chemotherapy. In some embodiments, G-CSF (if short acting G-CSF is used, then the first dose of G-CSF) is administered at least 24 h after the chemotherapy administration.

In some embodiments, the plinabulin and G-CSF are administered after the docetaxel administration. In some embodiments, the plinabulin is administered about 1 min, 5 min, 10 min, 15 min, 20 min, 25 min, 30 min, 45 min, 50 min, 1 h, 75 min, 1.5 h, 2 h, 2.5 h, 3 h, 4 h, 5 h, 6 h, 7 h, 8 h, 9 h, 10 h, 11 h, or 12 h after the administration of docetaxel. In some embodiments, the plinabulin is administered in less than about 1 min, 5 min, 10 min, 15 min, 20 min, 25 min, 30 min, 45 min, 50 min, 1 h, 75 min, 1.5 h, 2 h, 2.5 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, or 24 h after the administration of docetaxel. In some embodiments, the plinabulin is administered in more than about 1 min, 5 min, 10 min, 15 min, 20 min, 25 min, 30 min, 45 min, 50 min, 1 h, 75 min, 1.5 h, 2 h, 2.5 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, or 24 h after the administration of docetaxel. In some embodiments, the plinabulin is administered in about 1 min-5 min, 1 min-10 min, 1 min-15 min, 1 min-20 min, 1 min-25 min, 1 min-30 min, 1 min-45 min, 1 min-1 h, 1 min-75 min, 1 min-90 min, 1 min-120 min, 0.25 h-0.5 h, 0.25-0.75 h, 15 min-45 min, 15 min-75 min, 15 min-90 min, 15 min-120 min, 0.25-1 h, 30 min-45 min, 30 min-75 min, 30 min-90 min, 0.5 h-1 h, 0.5 h-2 h, 0.5 h-2.5 h, 1 h-2 h, 1 h-3 h, 1 h-5 h after the administration of docetaxel. In some embodiments, plinabulin is administered 30 mins after the docetaxel administration. In some embodiments, plinabulin is administered in less than 1 hour after the docetaxel administration.

In some embodiments, when plinabulin and G-CSF is co-administered prior to the chemotherapy administration, the plinabulin is administered about 1 min-5 min, 1 min-10 min, 1 min-15 min, 1 min-20 min, 1 min-25 min, 1 min-30 min, 0.25 h-0.5 h, 0.25-0.75 h, 0.25-1 h, 0.5 h-1 h, 0.5 h-2 h, 0.5 h-2.5 h, 1 h-2 h, 1 h-3 h, 1 h-5 h before the administration of the chemotherapy. In some embodiments, the plinabulin is administered about 1 min, 5 min, 10 min, 15 min, 20 min, 25 min, 30 min, 1 h, 1.5 h, 2 h, 2.5 h, 3 h, 4 h, 5 h, 6 h, 7 h, 8 h, 9 h, 10 h, 11 h, or 12 h before the administration of the chemotherapy. In some embodiments, the plinabulin is administered in less than about 1 min, 5 min, 10 min, 15 min, 20 min, 25 min, 30 min, 1 h, 1.5 h, 2 h, 2.5 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, or 24 h before the administration of the chemotherapy. In some embodiments, the plinabulin is administered in more than about 1 min, 5 min, 10 min, 15 min, 20 min, 25 min, 30 min, 1 h, 1.5 h, 2 h, 2.5 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, or 24 h before the administration of the chemotherapy.

In some embodiments, when plinabulin and G-CSF is co-administered prior to docetaxel administration, the plinabulin is administered about 1 min-5 min, 1 min-10 min, 1 min-15 min, 1 min-20 min, 1 min-25 min, 1 min-30 min, 0.25 h-0.5 h, 0.25-0.75 h, 0.25-1 h, 0.5 h-1 h, 0.5 h-2 h, 0.5 h-2.5 h, 1 h-2 h, 1 h-3 h, 1 h-5 h before the administration of docetaxel. In some embodiments, the plinabulin or G-CSF is administered about 1 min, 5 min, 10 min, 15 min, 20 min, 25 min, 30 min, 1 h, 1.5 h, 2 h, 2.5 h, 3 h, 4 h, 5 h, 6 h, 7 h, 8 h, 9 h, 10 h, 11 h, or 12 h before the administration of docetaxel. In some embodiments, the plinabulin or G-CSF is administered in less than about 1 min, 5 min, 10 min, 15 min, 20 min, 25 min, 30 min, 1 h, 1.5 h, 2 h, 2.5 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, or 24 h before the administration of docetaxel. In some embodiments, the plinabulin or G-CSF is administered in more than about 1 min, 5 min, 10 min, 15 min, 20 min, 25 min, 30 min, 1 h, 1.5 h, 2 h, 2.5 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, or 24 h before the administration of docetaxel.

In some embodiments, the infusion time for plinabulin is about 1 min, 5 min, 10 min, 15 min, 20 min, 25 min, 30 min, 45 min, 50 min, 1 h, 75 min, 1.5 h, 2 h, 2.5 h, 3 h, 4 h, 5 h, 6 h, 7 h, 8 h, 9 h, 10 h, 11 h, or 12 h. In some embodiments, the infusion time for plinabulin is less than about 1 min, 5 min, 10 min, 15 min, 20 min, 25 min, 30 min, 45 min, 50 min, 1 h, 75 min, 1.5 h, 2 h, 2.5 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, or 24 h after. In some embodiments, the infusion time for plinabulin is greater than about 1 min, 5 min, 10 min, 15 min, 20 min, 25 min, 30 min, 45 min, 50 min, 1 h, 75 min, 1.5 h, 2 h, 2.5 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, or 24 h. In some embodiments, the infusion time for plinabulin is about 1 min-5 min, 1 min-10 min, 1 min-15 min, 1 min-20 min, 1 min-25 min, 1 min-30 min, 1 min-45 min, 1 min-1 h, 1 min-75 min, 1 min-90 min, 1 min-120 min, 0.25 h-0.5 h, 0.25-0.75 h, 15 min-45 min, 15 min-75 min, 15 min-90 min, 15 min-120 min, 0.25-1 h, 30 min-45 min, 30 min-75 min, 30 min-90 min, 0.5 h-1 h, 0.5 h-2 h, 0.5 h-2.5 h, 1 h-2 h, 1 h-3 h, 1 h-5 h. In some embodiments, the infusion time for plinabulin is 30 mins for a single dose (e.g., 5, 10, 20, or less than 30 mg/m²). In some embodiments, the infusion time for plinabulin is about 1 hour (e.g., 20, 30, or greater than 30 mg/m²).

In some embodiments, when plinabulin is co-administered with G-CSF, the treatment schedule includes administration of the chemotherapy followed by the administration of plinabulin once every 3 weeks. In some embodiments, the treatment schedule includes administration of the chemotherapy followed by the administration of plinabulin about 30 mins after the chemotherapy administration, and the plinabulin is administered once every 3 weeks in a treatment cycle. In some embodiments, the treatment schedule includes administration of the chemotherapy followed by the administration of plinabulin once every week, 2 weeks, 3 weeks, 4 weeks, 5 weeks, 6 weeks, 7 weeks, or 8 weeks. In some embodiments, the treatment schedule includes administration of the chemotherapy followed by the administration of plinabulin two times every 1 week, 2 weeks, 3 weeks, 4 weeks, 5 weeks, 6 weeks, 7 weeks, or 8 weeks. In some embodiments, the treatment schedule includes administration of the chemotherapy followed by the administration of plinabulin once every week in a treatment cycle of 1 week, 2 weeks, 3 weeks, 4 weeks, 5 weeks, 6 weeks, 7 weeks, or 8 weeks. In some embodiments, the treatment schedule includes administration of the chemotherapy followed by the administration of plinabulin twice every 1 week in a treatment cycle of 1 week, 2 weeks, 3 weeks, 4 weeks, 5 weeks, 6 weeks, 7 weeks, or 8 weeks. In some embodiments, the treatment schedule includes administration of the chemotherapy followed by the administration of plinabulin on day 1, day 8, and day 15 of a 21-day treatment cycle. In some embodiments, the treatment schedule includes administering plinabulin following every dose of the chemotherapy administration. In some embodiments, the treatment schedule includes administering plinabulin following the initial dose/cycle of the chemotherapy administration and then administering plinabulin following every two doses, three doses, four doses, five doses, or six doses of the chemotherapy administration. In some embodiments, the treatment schedule includes administering plinabulin following every other dose of the chemotherapy administration. In some embodiments, the plinabulin is administered after every two doses, every three doses, every four doses, every five doses, or every six doses of the chemotherapy administration.

In some embodiments, the first dose of plinabulin or G-CSF is administered as soon as suspected or confirmed neutropenia development.

In some embodiments, when plinabulin is co-administered with G-CSF, the treatment schedule includes administration of the chemotherapy (e.g., docetaxel, TAC, or TC) followed by the administration of plinabulin once every 3 weeks. In some embodiments, the treatment schedule includes administration of the chemotherapy (e.g., docetaxel, TAC, or TC) followed by the administration of plinabulin about 30 mins after the chemotherapy administration, and the plinabulin is administered once every 3 weeks in a treatment cycle. In some embodiments, the treatment schedule includes administration of the chemotherapy (e.g., docetaxel, TAC, or TC) followed by the administration of plinabulin once every 1 week, 2 weeks, 3 weeks, 4 weeks, 5 weeks, 6 weeks, 7 weeks, or 8 weeks. In some embodiments, the treatment schedule includes administration of the chemotherapy (e.g., docetaxel, TAC, or TC) followed by the administration of plinabulin two times every 1 week, 2 weeks, 3 weeks, 4 weeks, 5 weeks, 6 weeks, 7 weeks, or 8 weeks. In some embodiments, the treatment schedule includes administration of the chemotherapy (e.g., docetaxel, TAC, or TC) followed by the administration of plinabulin once every 1 week in a treatment cycle of 1 week, 2 weeks, 3 weeks, 4 weeks, 5 weeks, 6 weeks, 7 weeks, or 8 weeks. In some embodiments, the treatment schedule includes administration of the chemotherapy (e.g., docetaxel, TAC, or TC) followed by the administration of plinabulin twice every 1 week in a treatment cycle of 1 week, 2 weeks, 3 weeks, 4 weeks, 5 weeks, 6 weeks, 7 weeks, or 8 weeks. In some embodiments, the treatment schedule includes administration of the chemotherapy (e.g., docetaxel, TAC, or TC) followed by the administration of plinabulin on day 1, day 8, and day 15 of a 21-day treatment cycle. In some embodiments, the treatment schedule includes co-administering plinabulin and G-CSF following every dose of the chemotherapy (e.g., docetaxel, TAC, or TC) administration. In some embodiments, the treatment schedule includes co-administering plinabulin and G-CSF following the initial dose of the chemotherapy (e.g., docetaxel, TAC, or TC) administration and then co-administering plinabulin and G-CSF following every two doses, three doses, four doses, five doses, or six doses of the chemotherapy administration. In some embodiments, the treatment schedule includes co-administering plinabulin and G-CSF following every other dose of the chemotherapy (e.g., docetaxel, TAC, or TC) administration. In some embodiments, the plinabulin and/or G-CSF is administered after every two doses, every three doses, every four doses, every five doses, or every six doses of the chemotherapy (e.g., docetaxel, TAC, or TC) administration.

In some embodiments, the treatment schedule includes administration of the chemotherapy (e.g., docetaxel, TAC, or TC) followed by the co-administration of plinabulin and G-CSF once every 3 weeks. In some embodiments, the treatment schedule includes administration of the chemotherapy (e.g., docetaxel, TAC, or TC) followed by the co-administration of plinabulin and G-CSF, and the plinabulin is administered once every 3 weeks in a treatment cycle. In some embodiments, the treatment schedule includes administration of the chemotherapy (e.g., docetaxel, TAC, or TC) followed by the co-administration of plinabulin and G-CSF once every 1 week, 2 weeks, 3 weeks, 4 weeks, 5 weeks, 6 weeks, 7 weeks, or 8 weeks. In some embodiments, the treatment schedule includes administration of the chemotherapy (e.g., docetaxel, TAC, or TC) followed by the administration of co-administration of plinabulin and G-CSF two times every 1 week, 2 weeks, 3 weeks, 4 weeks, 5 weeks, 6 weeks, 7 weeks, or 8 weeks. In some embodiments, the treatment schedule includes administration of the chemotherapy (e.g., docetaxel, TAC, or TC) followed by the co-administration of plinabulin and G-CSF once every 1 week in a treatment cycle of 1 week, 2 weeks, 3 weeks, 4 weeks, 5 weeks, 6 weeks, 7 weeks, or 8 weeks. In some embodiments, the treatment schedule includes administration of the chemotherapy (e.g., docetaxel, TAC, or TC) followed by the co-administration of plinabulin and G-CSF twice every 1 week in a treatment cycle of 1 week, 2 weeks, 3 weeks, 4 weeks, 5 weeks, 6 weeks, 7 weeks, or 8 weeks. In some embodiments, the treatment schedule includes administration of the chemotherapy (e.g., docetaxel, TAC, or TC) followed by the co-administration of plinabulin and G-CSF on day 1, day 8, and day 15 of a 21-day treatment cycle. In some embodiments, the treatment schedule includes co-administering plinabulin and G-CSF following every dose of the chemotherapy (e.g., docetaxel, TAC, or TC) administration. In some embodiments, the treatment schedule includes co-administering plinabulin and G-CSF following the initial dose of the chemotherapy (e.g., docetaxel, TAC, or TC) administration and then administering plinabulin following every two doses, three doses, four doses, five doses, or six doses of the chemotherapy administration. In some embodiments, the treatment schedule includes co-administering plinabulin and G-CSF following every other dose of the chemotherapy (e.g., docetaxel, TAC, or TC) administration. In some embodiments, the plinabulin and G-CSF are administered after every two doses, every three doses, every four doses, every five doses, or every six doses of the chemotherapy (e.g., docetaxel, TAC, or TC) administration.

In some embodiments, co-administering plinabulin and G-CSF includes administering only one dose G-CSF after the initial dose of plinabulin is administered and no G-CSF administration after the second dose of plinabulin per chemotherapy treatment cycle. In some embodiments, co-administering plinabulin and G-CSF includes administering one dose G-CSF after each dose plinabulin is administered. In some embodiments, co-administering plinabulin and G-CSF includes administering plinabulin once and then administering G-CSF daily for up to one week, two weeks, or three weeks, or until the patient's ANC returns to a level acceptable for the chemotherapy.

In some embodiments, the treatment schedule includes co-administering plinabulin and G-CSF following every cycle of the chemotherapy (e.g., docetaxel, TAC, or TC) administration. In some embodiments, the treatment schedule includes administering plinabulin following the initial cycle of the chemotherapy (e.g., docetaxel, TAC, or TC) administration and then administering plinabulin following every two cycles, three cycles, four cycles, five cycles, or six cycles of the chemotherapy administration. In some embodiments, the treatment schedule includes administering plinabulin following every other cycle of the chemotherapy (e.g., docetaxel, TAC, or TC) administration. In some embodiments, the plinabulin is administered after every two cycles, every three cycles, every four cycles, every five cycles, or every six cycles of the chemotherapy (e.g., docetaxel, TAC, or TC) administration.

The treatment cycle can be repeated as long as the regimen is clinically tolerated. In some embodiments, the treatment cycle for docetaxel or other chemotherapy is repeated for n times, wherein n is an integer in the range of 2 to 30. In some embodiments, n is 2, 3, 4, 5, 6, 7, 8, 9, or 10. In some embodiments, a new treatment cycle can occur immediately after the completion of the previous treatment cycle. In some embodiments, a new treatment cycle can occur a period of time after the completion of the previous treatment cycle.

In some embodiments, the co-administration of plinabulin and G-CSF can reduce the incidence of Grade 3 and/or 4 neutropenia by at least about 1%, 2%, 3%, 4%, 5%, 10%, 12.5%, 15%, 17.5%, 20%, 22.5%, 25%, 27.5%, 30%, 32.5%, 35%, 37.5%, 40%, 42.5%, 45%, 47.5%, 50%, 52.5%, 55%, 57.5%, 60%, 62.5%, 65%, 67.5%, 70%, 72.5%, 75%, 77.5%, 80%, 82.5%, 85%, 87.5%, 90%, 95%, or 100%. In some embodiments, the co-administration of plinabulin and G-CSF can reduce the incidence of Grade 3 and/or 4 neutropenia by at least about 5%, 10%, 12.5%, 15%, 17.5%, 20%, 22.5%, 25%, 27.5%, 30%, 32.5%, 35%, 37.5%, 40%, 42.5%, 45%, 47.5%, 50%, 52.5%, 55%, 57.5%, 60%, 62.5%, 65%, 67.5%, 70%, 72.5%, 75%, 77.5%, 80%, 82.5%, 85%, 87.5%, 90%, 95%, or 100%. In some embodiments, the co-administration of plinabulin and G-CSF can reduce the incidence of Grade 3 and/or 4 neutropenia by less than about 5%, 10%, 12.5%, 15%, 17.5%, 20%, 22.5%, 25%, 27.5%, 30%, 32.5%, 35%, 37.5%, 40%, 42.5%, 45%, 47.5%, 50%, 52.5%, 55%, 57.5%, 60%, 62.5%, 65%, 67.5%, 70%, 72.5%, 75%, 77.5%, 80%, 82.5%, 85%, 87.5%, 90%, 95%, or 100%. In some embodiments, the co-administration of plinabulin and G-CSF can reduce the incidence of Grade 3 and/or 4 neutropenia in the range of about 1%-5%, 1%-10%, 1%-15%, 1%-20%, 1%-30%, 1%-40%, 1%-50%, 2.5%-10%, 2.5%-15%, 2.5%-20%, 2.5%-30%, 5%-10%, 5%-15%, 5%-20%, 5%-30%, 5%-40%, 10%-40%, 12.5%-40%, 5%-50%, 10%-50%, 12.5%-50%, 15%-50%, 17.5%-50%, 20%-50%, 25%-50%, 27.5%-50%, 30%-50%, 5%-60%, 10%-60%, 12.5%-60%, 15%-60%, 17.5%-60%, 20%-60%, 25%-60%, 27.5%-60%, 30%-60%, 35%-60%, 37.5%-60%, 40%-60%, 45%-70%, or 50%-80%.

In some embodiments, the co-administration of plinabulin and G-CSF can be about 10%, 20%, 30%, 40%, 50%, 60%, 70%, 80%, 90%, 100%, 110%, 120%, 130%, 140%, 150%, 160%, 170%, 180%, 190%, 200%, 225%, 250%, 275%, 300%, 350% 400%, 450%, or 500% more effective than the use of G-CSF (e.g., pegfilgrastim) in reducing the incidence of Grade 3 and/or 4 neutropenia. In some embodiments, the co-administration of plinabulin and G-CSF can be greater than about 10%, 20%, 30%, 40%, 50%, 60%, 70%, 80%, 90%, 100%, 110%, 120%, 130%, 140%, 150%, 160%, 170%, 180%, 190%, 200%, 225%, 250%, 275%, 300%, 350% 400%, 450%, or 500% more effective than the use of G-CSF (e.g., pegfilgrastim) in reducing the incidence of Grade 3 and/or 4 neutropenia. In some embodiments, the co-administration of plinabulin and G-CSF can be less than about 10%, 20%, 30%, 40%, 50%, 60%, 70%, 80%, 90%, 100%, 110%, 120%, 130%, 140%, 150%, 160%, 170%, 180%, 190%, 200%, 225%, 250%, 275%, 300%, 350% 400%, 450%, or 500% more effective than the use of G-CSF (e.g., pegfilgrastim) in reducing the incidence of Grade 3 and/or 4 neutropenia. In some embodiments, the co-administration of plinabulin and G-CSF can be greater than about 10%, 20%, 30%, 40%, 50%, 60%, 70%, 80%, 90%, 100%, 110%, 120%, 130%, 140%, 150%, 160%, 170%, 180%, 190%, 200%, 225%, 250%, 275%, 300%, 350% 400%, 450%, or 500% more effective than the use of G-CSF (e.g., pegfilgrastim) in reducing the incidence of Grade 3 and/or 4 neutropenia.

In some embodiments, the co-administration of plinabulin and G-CSF can reduce the duration of severe neutropenia by about 1%, 2%, 3%, 4%, 5%, 10%, 12.5%, 15%, 17.5%, 20%, 22.5%, 25%, 27.5%, 30%, 32.5%, 35%, 37.5%, 40%, 42.5%, 45%, 47.5%, 50%, 52.5%, 55%, 57.5%, 60%, 62.5%, 65%, 67.5%, 70%, 72.5%, 75%, 77.5%, 80%, 82.5%, 85%, 87.5%, 90%, 95%, 100%, 110%, 120%, 130%, 140%, 150%, 160%, 170%, 180%, 190%, 200%, 225%, 250%, 275%, 300%, 350% 400%, 450%, 500%, 600%, 700%, 800%, 900%, 10 times, 11 times, 12 times, 13 times, 14 times, 15 times, or 16 times. In some embodiments, the co-administration of plinabulin and G-CSF can reduce the duration of severe neutropenia by greater than about 1%, 2%, 3%, 4%, 5%, 10%, 12.5%, 15%, 17.5%, 20%, 22.5%, 25%, 27.5%, 30%, 32.5%, 35%, 37.5%, 40%, 42.5%, 45%, 47.5%, 50%, 52.5%, 55%, 57.5%, 60%, 62.5%, 65%, 67.5%, 70%, 72.5%, 75%, 77.5%, 80%, 82.5%, 85%, 87.5%, 90%, 95%, 100%, 110%, 120%, 130%, 140%, 150%, 160%, 170%, 180%, 190%, 200%, 225%, 250%, 275%, 300%, 350% 400%, 450%, 500%, 600%, 700%, 800%, 900%, 10 times, 11 times, 12 times, 13 times, 14 times, 15 times, or 16 times. In some embodiments, the co-administration of plinabulin and G-CSF can reduce the duration of severe neutropenia by less than about 5%, 10%, 12.5%, 15%, 17.5%, 20%, 22.5%, 25%, 27.5%, 30%, 32.5%, 35%, 37.5%, 40%, 42.5%, 45%, 47.5%, 50%, 52.5%, 55%, 57.5%, 60%, 62.5%, 65%, 67.5%, 70%, 72.5%, 75%, 77.5%, 80%, 82.5%, 85%, 87.5%, 90%, 95%, 100%, 110%, 120%, 130%, 140%, 150%, 160%, 170%, 180%, 190%, 200%, 225%, 250%, 275%, 300%, 350% 400%, 450%, 500%, 600%, 700%, 800%, 900%, 10 times, 11 times, 12 times, 13 times, 14 times, 15 times, or 16 times. In some embodiments, the co-administration of plinabulin and G-CSF can reduce the duration of severe neutropenia in the range of about 5%-10%, 5%-20%, 5%-30%, 5%-40%, 5%-50%, 5%-60%, 5%-70%, 5%-80%, 5%-100%, 5%-2 times, 5%-5 times, 5%-15 times, 20%-10 times, or 50%-500%.

In some embodiments, the co-administration of plinabulin and G-CSF can be about 5%, 10%, 12.5%, 15%, 17.5%, 20%, 22.5%, 25%, 27.5%, 30%, 32.5%, 35%, 37.5%, 40%, 42.5%, 45%, 47.5%, 50%, 52.5%, 55%, 57.5%, 60%, 62.5%, 65%, 67.5%, 70%, 72.5%, 75%, 77.5%, 80%, 82.5%, 85%, 87.5%, 90%, 95%, 100%, 110%, 120%, 130%, 140%, 150%, 160%, 170%, 180%, 190%, 200%, 225%, 250%, 275%, 300%, 350% 400%, 450%, 500%, 600%, 700%, 800%, 900%, 10 times, 11 times, 12 times, 13 times, 14 times, 15 times, or 16 times more effective than G-CSF (e.g., pegfilgrastim) in reducing the duration of severe neutropenia. In some embodiments, the co-administration of plinabulin and G-CSF can be greater than about 5%, 10%, 12.5%, 15%, 17.5%, 20%, 22.5%, 25%, 27.5%, 30%, 32.5%, 35%, 37.5%, 40%, 42.5%, 45%, 47.5%, 50%, 52.5%, 55%, 57.5%, 60%, 62.5%, 65%, 67.5%, 70%, 72.5%, 75%, 77.5%, 80%, 82.5%, 85%, 87.5%, 90%, 95%, 100%, 110%, 120%, 130%, 140%, 150%, 160%, 170%, 180%, 190%, 200%, 225%, 250%, 275%, 300%, 350% 400%, 450%, 500%, 600%, 700%, 800%, 900%, 10 times, 11 times, 12 times, 13 times, 14 times, 15 times, or 16 times more effective than G-CSF (e.g., pegfilgrastim) in reducing the duration of severe neutropenia. In some embodiments, the co-administration of plinabulin and G-CSF can be less than about 5%, 10%, 12.5%, 15%, 17.5%, 20%, 22.5%, 25%, 27.5%, 30%, 32.5%, 35%, 37.5%, 40%, 42.5%, 45%, 47.5%, 50%, 52.5%, 55%, 57.5%, 60%, 62.5%, 65%, 67.5%, 70%, 72.5%, 75%, 77.5%, 80%, 82.5%, 85%, 87.5%, 90%, 95%, 100%, 110%, 120%, 130%, 140%, 150%, 160%, 170%, 180%, 190%, 200%, 225%, 250%, 275%, 300%, 350% 400%, 450%, 500%, 600%, 700%, 800%, 900%, 10 times, 11 times, 12 times, 13 times, 14 times, 15 times, or 16 times more effective than G-CSF (e.g., pegfilgrastim) in reducing the duration of severe neutropenia. In some embodiments, the co-administration of plinabulin and G-CSF can be in the range of about 5%-15 times, 20%-10 times, or 50%-500% more effective than G-CSF (e.g., pegfilgrastim) in reducing the duration of severe neutropenia.

Plinabulin and G-CSF can be co-administered following the chemotherapy to treat or ameliorate neutropenia. In some embodiments, a single dose of G-CSF (e.g., pegfilgrastim or eflapegrastim, or other long acting G-CSF) can be in the range of 0.1 mg to about 10 mg, about 0.3 mg to about 6 mg, about 0.1 mg to about 6 mg, 0.1 mg to about 7 mg, 0.5 mg to about 10 mg, from about 0.5 mg to about 8 mg, from about 0.5 mg to about 7 mg, from about 0.5 mg to about 6 mg, from about 0.5 mg to about 5 mg, from about 0.5 mg to about 4 mg, from about 0.5 mg to about 3 mg, 1 mg to about 100 mg, from about 1 mg to about 50 mg, from about 1 mg to about 25 mg, from about 1 mg to about 15 mg, from about 1 mg to about 10 mg, from about 1 mg to about 8 mg, from about 1 mg to about 7 mg, from about 1 mg to about 6 mg, from about 1 mg to about 5 mg, from about 1 mg to about 4 mg, from about 1 mg to about 3 mg, from about 2 mg to about 50 mg, from about 2 mg to about 25 mg, from about 2 mg to about 15 mg, from about 2 mg to about 10 mg, from about 2 mg to about 10 mg, from about 2 mg to about 8 mg, from about 2 mg to about 7 mg, from about 2 mg to about 6 mg, from about 2 mg to about 5 mg, from about 2 mg to about 4 mg, from about 2 mg to about 3 mg, from about 3 mg to about 50 mg, from about 3 mg to about 25 mg, from about 3 mg to about 15 mg, from about 3 mg to about 10 mg, from about 3 mg to about 10 mg, from about 3 mg to about 8 mg, from about 3 mg to about 7 mg, from about 3 mg to about 6 mg, from about 3 mg to about 5 mg, from about 3 mg to about 4 mg, from about 4 mg to about 50 mg, from about 4 mg to about 25 mg, from about 4 mg to about 15 mg, from about 4 mg to about 10 mg, from about 4 mg to about 6 mg, from about 4 mg to about 5 mg, from about 5 mg to about 25 mg, from about 5 mg to about 15 mg, from about 5 mg to about 10 mg, or from about 5 mg to about 8 mg. In some embodiments, a single dose of G-CSF (e.g., pegfilgrastim or eflapegrastim, or other long acting G-CSF) can have an amount equivalent to filgrastim in the range of 0.5 mg to about 10 mg, from about 0.5 mg to about 8 mg, from about 0.5 mg to about 7 mg, from about 0.5 mg to about 6 mg, from about 0.5 mg to about 5 mg, from about 0.5 mg to about 4 mg, from about 0.5 mg to about 3 mg, 1 mg to about 100 mg, from about 1 mg to about 50 mg, from about 1 mg to about 25 mg, from about 1 mg to about 15 mg, from about 1 mg to about 10 mg, from about 1 mg to about 8 mg, from about 1 mg to about 7 mg, from about 1 mg to about 6 mg, from about 1 mg to about 5 mg, from about 1 mg to about 4 mg, from about 1 mg to about 3 mg, from about 2 mg to about 50 mg, from about 2 mg to about 25 mg, from about 2 mg to about 15 mg, from about 2 mg to about 10 mg, from about 2 mg to about 10 mg, from about 2 mg to about 8 mg, from about 2 mg to about 7 mg, from about 2 mg to about 6 mg, from about 2 mg to about 5 mg, from about 2 mg to about 4 mg, from about 2 mg to about 3 mg, from about 3 mg to about 50 mg, from about 3 mg to about 25 mg, from about 3 mg to about 15 mg, from about 3 mg to about 10 mg, from about 3 mg to about 10 mg, from about 3 mg to about 8 mg, from about 3 mg to about 7 mg, from about 3 mg to about 6 mg, from about 3 mg to about 5 mg, from about 3 mg to about 4 mg, from about 4 mg to about 50 mg, from about 4 mg to about 25 mg, from about 4 mg to about 15 mg, from about 4 mg to about 10 mg, from about 4 mg to about 6 mg, from about 4 mg to about 5 mg, from about 5 mg to about 25 mg, from about 5 mg to about 15 mg, from about 5 mg to about 10 mg, or from about 5 mg to about 8 mg. In some embodiments, a single dose of G-CSF (e.g., pegfilgrastim, eflapegrastim, or other long acting G-CSF) may be from about 3 mg to about 10 mg, or from about 4 mg to about 8 mg, or equivalent to an amount of filgrastim in the ranges herein described. In some embodiments, a single dose of G-CSF (e.g., pegfilgrastim, eflapegrastim, or other long acting G-CSF) may be greater than about 0.1 mg, about 0.2 mg, about 0.3 mg, about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 7 mg, about 8 mg, about 9 mg, or about 10 mg. In some embodiments, a single dose of G-CSF (e.g., pegfilgrastim, eflapegrastim, or other long acting G-CSF) may be less than about 1 mg, about 1.5 mg, about 2 mg, about 2.5 mg, about 3 mg, about 3.5 mg, about 4 mg, about 4.5 mg, about 5 mg, about 5.5 mg, about 6 mg, about 7 mg, about 8 mg, about 9 mg, about 10 mg, about 12.5 mg, or about 15 mg. In some embodiments, a single dose of G-CSF (e.g., pegfilgrastim, eflapegrastim, or other long acting G-CSF) may be about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 7 mg, about 8 mg, about 9 mg, or about 10 mg. In some embodiments, a single dose of G-CSF (e.g., pegfilgrastim, eflapegrastim, or other long acting G-CSF) may be about 6 mg.

In some embodiments, the total dosage of G-CSF (e.g., pegfilgrastim or eflapegrastim, or other long acting G-CSF) administered in a 21-day treatment cycle can be in the range of 0.1 mg to about 10 mg, about 0.3 mg to about 6 mg, about 0.1 mg to about 6 mg, 0.1 mg to about 7 mg, 0.5 mg to about 10 mg, from about 0.5 mg to about 8 mg, from about 0.5 mg to about 7 mg, from about 0.5 mg to about 6 mg, from about 0.5 mg to about 5 mg, from about 0.5 mg to about 4 mg, from about 0.5 mg to about 3 mg, 1 mg to about 100 mg, from about 1 mg to about 50 mg, from about 1 mg to about 25 mg, from about 1 mg to about 15 mg, from about 1 mg to about 10 mg, from about 1 mg to about 8 mg, from about 1 mg to about 7 mg, from about 1 mg to about 6 mg, from about 1 mg to about 5 mg, from about 1 mg to about 4 mg, from about 1 mg to about 3 mg, from about 2 mg to about 50 mg, from about 2 mg to about 25 mg, from about 2 mg to about 15 mg, from about 2 mg to about 10 mg, from about 2 mg to about 10 mg, from about 2 mg to about 8 mg, from about 2 mg to about 7 mg, from about 2 mg to about 6 mg, from about 2 mg to about 5 mg, from about 2 mg to about 4 mg, from about 2 mg to about 3 mg, from about 3 mg to about 50 mg, from about 3 mg to about 25 mg, from about 3 mg to about 15 mg, from about 3 mg to about 10 mg, from about 3 mg to about 10 mg, from about 3 mg to about 8 mg, from about 3 mg to about 7 mg, from about 3 mg to about 6 mg, from about 3 mg to about 5 mg, from about 3 mg to about 4 mg, from about 4 mg to about 50 mg, from about 4 mg to about 25 mg, from about 4 mg to about 15 mg, from about 4 mg to about 10 mg, from about 4 mg to about 6 mg, from about 4 mg to about 5 mg, from about 5 mg to about 25 mg, from about 5 mg to about 15 mg, from about 5 mg to about 10 mg, or from about 5 mg to about 8 mg. In some embodiments, the total dosage of G-CSF (e.g., pegfilgrastim or eflapegrastim, or other long acting G-CSF) per 21-day cycle can have an amount equivalent to filgrastim in the range of 0.5 mg to about 10 mg, from about 0.5 mg to about 8 mg, from about 0.5 mg to about 7 mg, from about 0.5 mg to about 6 mg, from about 0.5 mg to about 5 mg, from about 0.5 mg to about 4 mg, from about 0.5 mg to about 3 mg, 1 mg to about 100 mg, from about 1 mg to about 50 mg, from about 1 mg to about 25 mg, from about 1 mg to about 15 mg, from about 1 mg to about 10 mg, from about 1 mg to about 8 mg, from about 1 mg to about 7 mg, from about 1 mg to about 6 mg, from about 1 mg to about 5 mg, from about 1 mg to about 4 mg, from about 1 mg to about 3 mg, from about 2 mg to about 50 mg, from about 2 mg to about 25 mg, from about 2 mg to about 15 mg, from about 2 mg to about 10 mg, from about 2 mg to about 10 mg, from about 2 mg to about 8 mg, from about 2 mg to about 7 mg, from about 2 mg to about 6 mg, from about 2 mg to about 5 mg, from about 2 mg to about 4 mg, from about 2 mg to about 3 mg, from about 3 mg to about 50 mg, from about 3 mg to about 25 mg, from about 3 mg to about 15 mg, from about 3 mg to about 10 mg, from about 3 mg to about 10 mg, from about 3 mg to about 8 mg, from about 3 mg to about 7 mg, from about 3 mg to about 6 mg, from about 3 mg to about 5 mg, from about 3 mg to about 4 mg, from about 4 mg to about 50 mg, from about 4 mg to about 25 mg, from about 4 mg to about 15 mg, from about 4 mg to about 10 mg, from about 4 mg to about 6 mg, from about 4 mg to about 5 mg, from about 5 mg to about 25 mg, from about 5 mg to about 15 mg, from about 5 mg to about 10 mg, or from about 5 mg to about 8 mg. In some embodiments, the total dosage of G-CSF (e.g., pegfilgrastim, eflapegrastim, or other long acting G-CSF) per 21-day cycle may be from about 3 mg to about 10 mg, or from about 4 mg to about 8 mg, or equivalent to an amount of filgrastim in the ranges herein described. In some embodiments, the total dosage of G-CSF (e.g., pegfilgrastim, eflapegrastim, or other long acting G-CSF) in a 21-day cycle may be greater than about 0.1 mg, about 0.2 mg, about 0.3 mg, about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 7 mg, about 8 mg, about 9 mg, or about 10 mg. In some embodiments, a total dosage of G-CSF (e.g., pegfilgrastim, eflapegrastim, or other long acting G-CSF) in a 21-day cycle may be less than about 1 mg, about 1.5 mg, about 2 mg, about 2.5 mg, about 3 mg, about 3.5 mg, about 4 mg, about 4.5 mg, about 5 mg, about 5.5 mg, about 6 mg, about 7 mg, about 8 mg, about 9 mg, about 10 mg, about 12.5 mg, or about 15 mg. In some embodiments, a total dosage of G-CSF (e.g., pegfilgrastim, eflapegrastim, or other long acting G-CSF) in a 21-day cycle may be about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 7 mg, about 8 mg, about 9 mg, or about 10 mg. In some embodiments, a total dosage of G-CSF (e.g., pegfilgrastim, eflapegrastim, or other long acting G-CSF) in a 21-day cycle may be about 6 mg.

In some embodiments, G-CSF (e.g., filgrastim or other short acting G-CSF) can be administered in an amount of about 1, 2, 3, 4, 5, 6, 7, 8, 9, 10, 11, 12, 13, 14, 15, 16, 17, 18, 19, 20 μg/kg/day. In some embodiments, G-CSF (e.g., filgrastim or other short acting G-CSF) can be administered in an amount greater than about 1, 2, 3, 4, 5, 6, 7, 8, 9, 10, 11, 12, 13, 14, 15, 16, 17, 18, 19, 20 μg/kg/day. In some embodiments, G-CSF (e.g., filgrastim or other short acting G-CSF) can be administered in an amount less than about 5, 6, 7, 8, 9, 10, 11, 12, 13, 14, 15, 16, 17, 18, 19, 20, 30, 40, or 50 μg/kg/day. In some embodiments, G-CSF (e.g., filgrastim or other short acting G-CSF) can be administered in an amount of about 1-5, 1-10, 1-15, 1-20, 1-30, 2.5-5, 2.5-7.5, 2.5-10, 2.5-15, 2.5-20, 5-10, 5-15, 5-20, 5-25, or 5-30 μg/kg/day. In some embodiments, the G-CSF is administered in an amount of about 0.05, 0.75, 0.1, 0.2, 0.3, 0.4, 0.48, 0.5, 0.6, 0.7, 0.8, 0.9, 1.0, 1.2, 1.5, 1.75, or 2 mg per dose. In some embodiments, the G-CSF is administered in the range of about 0.05-0.5 mg, 0.05-1.0 mg, 0.1-1.0 mg, or 0.05-2.0 mg per dose.

Administration of Plinabulin and G-CSF can help prevent or treat severe neutropenia induced by the chemotherapy treatment. In some embodiments, the G-CSF (e.g., pegfilgrastim or filgrastim) is administered once per chemotherapy cycle. In some embodiments, the G-CSF (e.g., pegfilgrastim or filgrastim) is not administered between 14 hours and 24 hours after the administration of cytotoxic chemotherapy. In some embodiments, the G-CSF (e.g., pegfilgrastim or filgrastim) is not administered before 24 hours after the administration of cytotoxic chemotherapy In some embodiments, two doses of G-CSF (e.g., pegfilgrastim or filgrastim) are administered one week apart. In some embodiments, the G-CSF (e.g., pegfilgrastim or filgrastim) is administered once, twice, three times, four times, five times, six times, seven times a week for one week or two weeks. In some embodiments, the G-CSF (e.g., pegfilgrastim or filgrastim) is administered once a week, every two weeks, every three weeks, every four weeks, every five weeks, or ever six weeks. In some embodiments, the first dose of G-CSF (e.g., pegfilgrastim or filgrastim) is administered as soon as the suspected or confirmed exposure to myelosuppressive chemotherapy or myelosuppressive dose of radiation. In some embodiments, the G-CSF (e.g., pegfilgrastim or filgrastim) is administered subcutaneously.

In some embodiments, during the chemotherapy treatment cycle, the chemotherapeutic agent(s) is only administered once at the beginning of the treatment cycle, followed by the co-administration of plinabulin and G-CSF once, twice, three times, four times, five times, or six times during the treatment cycle. In some embodiments, during the chemotherapy treatment cycle, the chemotherapeutic agent(s) is only administered once at the beginning of the treatment cycle, followed by the co-administration of plinabulin and G-CSF once every week, once every two weeks, once every three weeks, once every four weeks, once evert five weeks, or once every six weeks. In some embodiments, during the chemotherapy treatment cycle, the chemotherapeutic agent(s) is only administered once at the beginning of the treatment cycle, followed by the co-administration of plinabulin and G-CSF once a week, twice a week, three times per week, four times per week, five times per week, six times per week, or daily during a one-week, two-week, three-week, four-week, or five-week treatment cycle.

In some embodiments, the G-CSF and plinabulin can be co-administered or administered separately once every three weeks. In some embodiments, the G-CSF and plinabulin can be co-administered or administered separately once every week, once every two weeks, once every three weeks, once every four weeks, once evert five weeks, or once every six weeks. In some embodiments, the G-CSF and plinabulin can be co-administered or administered separately once a week. In some embodiments, G-CSF and plinabulin can be co-administered or administered separately once a week, twice a week, three times per week, four times per week, five times per week, six times per week, or daily during a one-week, two-week, three-week, four-week, or five-week treatment cycle. The administration can be on the same or different day of each week in the treatment cycle. In some embodiments, the plinabulin is administered prior to the G-CSF administration. In some embodiments, the plinabulin is administered concurrently with the G-CSF administration. In some embodiments, the plinabulin is administered after the G-CSF administration.

In some embodiments, the G-CSF is administered prior to the plinabulin administration. In some embodiments, the G-CSF is administered concurrently with the plinabulin administration. In some embodiments, the G-CSF is administered after the plinabulin administration. In some further embodiments, the G-CSF is administered about 10 hours to about 72 hours, about 20 hours to about 72 hours, about 20 hours to about 70 hours, about 20 hours to about 60 hours, about 20 hours to about 50 hours, about 20 to about 48 hours, about 20 hours to about 40 hours, about 20 hours to about 36 hours, about 20 hours to about 30 hours, or about 20 hours to about 24 hours after the plinabulin administration. In some yet further embodiments, the G-CSF is administered within 48 hours, within 24 hours, or within 12 hours after the administration of the plinabulin.

In some embodiments, the G-CSF (if a short acting G-CSF is used, then the first dose of G-CSF) is administered after about 6 h, 12 h, 18 h, 24 h, 36 g, 48 h, or 72 h after the administration of the chemotherapy. In some embodiments, the G-CSF (if a short acting G-CSF is used, then the first dose of G-CSF) is administered in less than about 12 h, 18 h, 24 h, 36 h, 48 h, 60 h, 72 h, 84 h, 96 h, 5 days, 6 days, or 7 days after the administration of the chemotherapy. In some embodiments, the G-CSF (if a short acting G-CSF is used, then the first dose of G-CSF) is administered in about 1 h-24 h, 12 h-36 h, 10 h-40 h, 24 hour to 36 hour, 1 day-2 days, 1 day-5 days, 1 day-1 week after the administration of the chemotherapy. In some embodiments, the G-CSF (if a short acting G-CSF is used, then the first dose of G-CSF) is administered at least about 24 h after the chemotherapy administration. In some embodiments, the G-CSF (if a short acting G-CSF is used, then the first dose of G-CSF) is administered at least about 48 h after the chemotherapy administration. In some embodiments, the G-CSF (if a short acting G-CSF is used, then the first dose of G-CSF) is administered about 24 h after the chemotherapy administration. In some embodiments, the G-CSF (if a short acting G-CSF is used, then the first dose of G-CSF) is administered about 48 h after the chemotherapy administration.

Some embodiments include administering one, two, three, four, five, six, or more cycles of a chemotherapy regimen, each cycle of the chemotherapy regimen independently comprising administering one or more chemotherapeutic agents on day 1, day 2, day 3, day 4, day 5, or a combination thereof, administering plinabulin on day 1, day 2, day 3, day 4, day 5, day 6, day 7 or a combination thereof, and administering one or more G-CSF drugs on day 2, day 3, day 4, day 5, day 6, day 7, day 8, day 9, day 10, day 11, day 12, day 13, day 14, or a combination thereof. In some embodiments, the one or more chemotherapeutic agents may be administered, independently in each cycle of the chemotherapy, according to the foregoing description or of any description elsewhere herein. In some embodiments, the plinabulin may be administered, independently in each cycle of the chemotherapy, according to the foregoing description or of any description elsewhere herein. In some embodiments, the one or more G-CSF drugs may be administered, independently in each cycle of the chemotherapy, according to the foregoing description or of any description elsewhere herein. In some embodiments, each cycle of the chemotherapy regimen lasts up to 30 days, lasts up to 21 days, lasts up to 14 days, or lasts up to 7 days. In some embodiments, each cycle of the chemotherapy regimen lasts 30 days, lasts 21 days, lasts 14 days, or lasts 7 days.

Administration of the pharmaceutical compositions described herein can be via any of the accepted modes of administration for agents that serve similar utilities including, but not limited to, orally, sublingually, buccally, subcutaneously, intravenously, intranasally, topically, transdermally, intradermally, intraperitoneally, intramuscularly, intrapulmonarilly, vaginally, rectally, or intraocularly. Oral and parenteral administrations are customary in treating the indications that are the subject of the preferred embodiments

### Pharmaceutical Composition

Some embodiments relate to a pharmaceutical composition including plinabulin. Some embodiments relate to a pharmaceutical composition comprising about 1 mg to about 100 mg of plinabulin.

In some embodiments, the compositions described herein can be administered or used in combination with docetaxel treatment.

Other embodiments include co-administering Plinabulin, G-CSF, and docetaxel in separate compositions or the same composition. Thus, some embodiments include a first pharmaceutical compositions comprising: (a) a safe and therapeutically effective amount of docetaxel or pharmaceutically acceptable salts thereof and (b) a pharmaceutically acceptable carrier, diluent, excipient or combination thereof; a second pharmaceutical composition comprising: (a) a safe and therapeutically effective amount of plinabulin and (b) a pharmaceutically acceptable carrier, diluent, excipient or combination thereof, and a third pharmaceutical composition comprising: (a) a safe and therapeutically effective amount of G-CSF and (b) a pharmaceutically acceptable carrier, diluent, excipient or combination thereof. Some embodiments include a pharmaceutical composition comprising: (a) a safe and therapeutically effective amount of docetaxel or pharmaceutically acceptable salts thereof; (b) a safe and therapeutically effective amount of plinabulin; (c) a safe and therapeutically effective amount of G-CSF, and (d) a pharmaceutically acceptable carrier, diluent, excipient or combination thereof.

In some embodiments, the pharmaceutical composition described herein can further include one or more pharmaceutically acceptable diluents. In some embodiments, the pharmaceutically acceptable diluent can include Kolliphor® (Polyethylene glycol (15)-hydroxystearate). In some embodiments, the pharmaceutically acceptable diluent can include propylene glycol. In some embodiments, the pharmaceutically acceptable diluents can include kolliphor (Kolliphor HS 15) and propylene glycol. In some embodiments, the pharmaceutically acceptable diluents can include kolliphor and propylene glycol, wherein the kolliphor is about 40% by weight and propylene glycol is about 60% by weight based on the total weight of the diluents. In some embodiments, the composition can further include one or more other pharmaceutically acceptable excipients.

Standard pharmaceutical formulation techniques can be used to make the pharmaceutical compositions described herein, such as those disclosed in Remington's The Science and Practice of Pharmacy, 21st Ed., Lippincott Williams & Wilkins (2005), incorporated herein by reference in its entirety. Accordingly, some embodiments include pharmaceutical compositions comprising: (a) a safe and therapeutically effective amount of Plinabulin or pharmaceutically acceptable salts thereof; and (b) a pharmaceutically acceptable carrier, diluent, excipient or combination thereof.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The compositions described herein are preferably provided in unit dosage form. As used herein, a “unit dosage form” is a composition containing an amount of a compound or composition that is suitable for administration to an animal, preferably a mammalian subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, although a single administration is not specifically excluded. The skilled artisan will recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, sublingual, buccal, nasal, rectal, topical (including transdermal and intradermal), ocular, intracerebral, intracranial, intrathecal, intra-arterial, intravenous, intramuscular, or other parental routes of administration. The skilled artisan will appreciate that oral and nasal compositions include compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the activity of the compound or composition. The amount of carrier employed in conjunction with the compound or composition is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules (e.g., liquid gel capsule and solid gel capsule), granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carriers suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, sucrose, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject composition is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort may be maximized as much as possible, although sometimes formulation considerations (e.g. drug stability) may necessitate less than optimal comfort. In the case that comfort cannot be maximized, the liquid may be formulated such that the liquid is tolerable to the patient for topical ophthalmic use. Additionally, an ophthalmically acceptable liquid may either be packaged for single use, or contain a preservative to prevent contamination over multiple uses.

For ophthalmic application, solutions or medicaments are often prepared using a physiological saline solution as a major vehicle. Ophthalmic solutions may preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

Ophthalmically acceptable antioxidants include, but are not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium (EDTA), although other chelating agents may also be used in place or in conjunction with it.

For topical use, creams, ointments, gels, solutions or suspensions, etc., containing the composition disclosed herein are employed. Topical formulations may generally be comprised of a pharmaceutical carrier, co-solvent, emulsifier, penetration enhancer, preservative system, and emollient.

For intravenous administration, the compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one or more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The actual dose of plinabulin described herein depends on the chemotherapeutic agent used; and on the condition to be treated; the selection of the appropriate dose is well within the knowledge of the skilled artisan. In some embodiments, a single dose of Plinabulin may be from about 5 mg/m² to about 150 mg/m² of body surface area, from about 5 mg/m² to about 100 mg/m² of body surface area, from about 10 mg/m² to about 100 mg/m² of body surface area, from about 10 mg/m² to about 80 mg/m² of body surface area, from about 10 mg/m² to about 50 mg/m² of body surface area, from about 10 mg/m² to about 40 mg/m² of body surface area, from about 10 mg/m² to about 30 mg/m² of body surface area, from about 13.5 mg/m² to about 100 mg/m² of body surface area, from about 13.5 mg/m² to about 80 mg/m² of body surface area, from about 13.5 mg/m² to about 50 mg/m² of body surface area, from about 13.5 mg/m² to about 40 mg/m² of body surface area, from about 13.5 mg/m² to about 30 mg/m² of body surface area, from about 15 mg/m² to about 80 mg/m² of body surface area, from about 15 mg/m² to about 50 mg/m² of body surface area, or from about 15 mg/m² to about 30 mg/m² of the body surface area. In some embodiments, a single dose of Plinabulin may be from about 13.5 mg/m² to about 30 mg/m² of body surface area. In some embodiments, a single dose of Plinabulin may be greater than about 5 mg/m², about 10 mg/m², about 12.5 mg/m², about 13.5 mg/m², about 15 mg/m², about 17.5 mg/m², about 20 mg/m², about 22.5 mg/m², about 25 mg/m², about 27.5 mg/m², about 30 mg/m², about 40 mg/m², about 50 mg/m², about 60 mg/m², about 70 mg/m², about 80 mg/m², about 90 mg/m², or about 100 mg/m², of the body surface area. In some embodiments, a single dose of Plinabulin may be less than about 5 mg/m², about 10 mg/m², about 12.5 mg/m², about 13.5 mg/m², about 15 mg/m², about 17.5 mg/m², about 20 mg/m², about 22.5 mg/m², about 25 mg/m², about 27.5 mg/m², about 30 mg/m², about 40 mg/m², about 50 mg/m², about 60 mg/m², about 70 mg/m², about 80 mg/m², about 90 mg/m², or about 100 mg/m², of body surface area. In some embodiments, a single dose of Plinabulin may be about 5 mg/m², about 10 mg/m², about 12.5 mg/m², about 13.5 mg/m², about 15 mg/m², about 17.5 mg/m², about 20 mg/m², about 22.5 mg/m², about 25 mg/m², about 27.5 mg/m², about 30 mg/m², about 40 mg/m², about 50 mg/m², about 60 mg/m², about 70 mg/m², about 80 mg/m², about 90 mg/m², or about 100 mg/m², of the body surface area.

Some embodiments relate to a composition comprising about 1 mg to 100 mg of plinabulin. In some embodiments, the composition includes about 5 mg to about 300 mg, from about 5 mg to about 200 mg, from about 7.5 mg to about 200 mg, from about 10 mg to about 100 mg, from about 15 mg to about 100 mg, from about 20 mg to about 100 mg, from about 30 mg to about 100 mg, from about 40 mg to about 100 mg, from about 10 mg to about 80 mg, from about 15 mg to about 80 mg, from about 20 mg to about 80 mg, from about 30 mg to about 80 mg, from about 40 mg to about 80 mg, from about 10 mg to about 60 mg, from about 15 mg to about 60 mg, from about 20 mg to about 60 mg, from about 30 mg to about 60 mg, or from about 40 mg to about 60 mg of plinabulin. In some embodiments, a single dose of Plinabulin or other therapeutic agent may be from about 20 mg to about 60 mg, from about 27 mg to about 60 mg, from about 20 mg to about 45 mg, or from about 27 mg to about 45 mg. In some embodiments, a single dose of Plinabulin or other therapeutic agent may be greater than about 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, or about 200 mg. In some embodiments, a single dose of Plinabulin or other therapeutic agent may be less than about 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, or about 200 mg.

Some embodiments relate to a pharmaceutical composition including plinabulin and one or more G-CSF drugs.

Some embodiments relate to a sterile container comprising the pharmaceutical composition of plinabulin described herein. Some embodiments relate to a kit including a chemotherapeutic agent, about 1 mg to about 80 mg of plinabulin, and about 0.1 mg to about 20 mg of G-CSF, wherein the chemotherapy, G-CSF, and the plinabulin are provided in separate sterile containers.

In some embodiments, the amount of plinabulin in the kit is less than 50 mg. In some embodiments, the amount of plinabulin in the sterile container is about 10 mg. In some embodiments, the amount of plinabulin in the sterile container is about 20 mg. In some embodiments, the amount of plinabulin in the sterile container is about 30 mg. In some embodiments, the amount of plinabulin in the sterile container is about 40 mg. In some embodiments, the amount of G-CSF in the sterile container is less than 10 mg. In some embodiments, the amount of G-CSF in the sterile container is about 6 mg. In some embodiments, the amount of G-CSF in the sterile container is about 3 mg. In some embodiments, the amount of G-CSF in the sterile container is about 1.5 mg.

Some embodiments include a kit comprising a plinabulin, G-CSF, and docetaxel. In one embodiment, plinabulin, G-CSF, and docetaxel are provided in separate sterile containers. In the case of solids for reconstitution, the agents may be added to the container simultaneously or may be dry-powder filled into the container in separate steps. In some embodiments, the solids are sterile crystalline products. In other embodiment, the solids are lyophiles.

### EXAMPLE

### Example 1

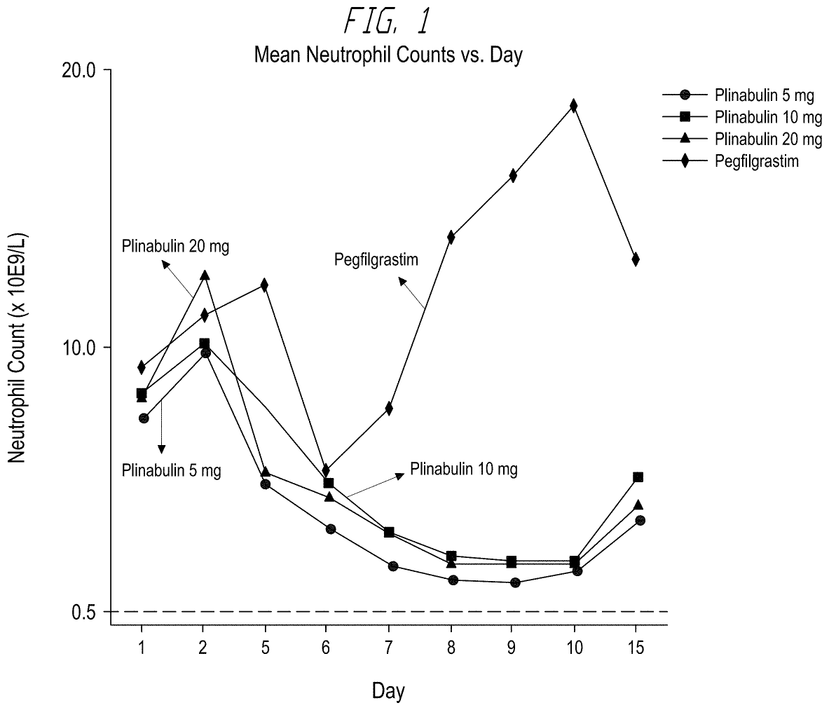

A randomized, double blind study to evaluate duration of severe neutropenia with plinabulin versus pegfilgrastim in patients with solid tumors receiving docetaxel myelosuppressive chemotherapy was performed. Patients were randomly assigned to the following arms (with the respective sample sizes): Arm 1: Docetaxel (75 mg/m²)+Pegfilgrastim (6 mg) (n=14); Arm 2: Docetaxel (75 mg/m²)+Plinabulin (20 mg/m²) (n=14); Arm 3: Docetaxel (75 mg/m²)+Plinabulin (10 mg/m²) (n=14); and Arm 4: Docetaxel (75 mg/m²)+Plinabulin (5 mg/m²) (n=13). The testing results are shown in FIG. 1.

As shown in FIG. 1, the neutrophil count in the pegfilgrastim group began to drop after 10 days, while the neutrophil count in the plinabulin groups started to rise again on day 10. The results showed that plinabulin was effective in treating neutropenia induced by chemotherapeutic agent. Plinabulin and Pegfilgrastim had different profile of reducing neutropenia, and the nadir time point was different for plinabulin versus pegfilgrastim, suggesting the two drugs can be used in combination to maintain the neutrophil count level continuously. Plinabulin prevented the overshot of the ANC during recovery. The results also suggested that the amount of G-CSF drug or plinabulin required for the neutropenia treatment may be decreased due to the supplemental effect of the combination.

### Example 2

A multicenter, randomized study, involving G-CSF and plinabulin was performed. The Phase 2 portion was randomized and open label. The decision to complete the Phase 2 portion of the study as open label was made to reduce the complexities of study conduct and to allow for the assessment, via QoL, of same-day plinabulin dosing (i.e on the day of chemotherapy dosing) versus next day dosing with G-CSF. Patients with first line breast cancer were enrolled in the study.

Patients received up to 4 cycles of a docetaxel/doxorubicin/cyclophosphamide based chemotherapy regimen, every 3 weeks (21 days). On Day 1 of Cycle 1, all patients receive docetaxel (75 mg/m²), doxorubicin (50 mg/m²), and cyclophosphamide (500 mg/m²)-Taxotere, Adriamycin and cyclophosphamide (TAC).

During Cycles 2 to 4, the doxorubicin component may be omitted at the discretion of the investigator, i.e., TC can be administered instead of TAC.

The eligibility of all patients was determined during a 28-day screening period.

Since plinabulin has demonstrated efficacy against docetaxel-induced neutropenia in humans, and since the beneficial effects of plinabulin also were demonstrated in non-clinical studies with the two other components of the TAC regimen, plinabulin helped ameliorate neutropenia induced by TAC.

Phase 2 (Open Label): In Phase 2, patients were randomly assigned to one of the treatment arms, with the arm designation and planned intervention as shown in Table 2.

TABLE 2
Treatments Administered for Phase 2

| | | Cycles 1 to 4, | |
|---|---|---|---|
| | | Day 1 21 Day | |
| | | Cycle 30 (±2) | Cycles 1 to 4, |
| | | minutes from | Day 2 21 Day |
| | Cycles 1 to 4, Day 1 21 Day | the end of the | Cycle ≥24 hours |
| | Cycle (TAC) | Docetaxel infusion | post Day 1 |
| Arm 1 | Pre-medication (up to 30 minutes) | No drug | Pegfilgrastim |
| | Doxorubicin (50 mg/m²) = | administered | (6.0 mg) SC |
| | Approximately 15 minute IV treatment | | single dose |
| | Cyclophosphamide (500 mg/m²) = | | |
| | Approximately 30 minute IV treatment | | |
| | Docetaxel (75 mg/m²) = | | |
| | Approximately 60 minute IV treatment | | |
| Arm 2 | Pre-medication (up to 30 minutes) | Plinabulin | pegfilgrastim |
| | Doxorubicin (50 mg/m²) = | (20 mg/m²) | (6.0 mg) |
| | Approximately 15 minute IV treatment | 30 minute IV | |
| | Cyclophosphamide (500 mg/m²) = | infusion | |
| | Approximately 30 minute IV treatment | | |
| | Docetaxel (75 mg/m²) = | | |
| | Approximately 60 minute IV treatment | | |
| Arm 3 | Pre-medication (up to 30 minutes) | Plinabulin | pegfilgrastim |
| | Doxorubicin (50 mg/m²) = | (20 mg/m²) | (3.0 mg) |
| | Approximately 15 minute IV treatment | 30 minute IV | |
| | Cyclophosphamide (500 mg/m²) = | infusion | |
| | Approximately 30 minute IV treatment | | |
| | Docetaxel (75 mg/m²) = | | |
| | Approximately 60 minute IV treatment | | |
| Arm 4 | Pre-medication (up to 30 minutes) | Plinabulin | pegfilgrastim |
| | Doxorubicin (50 mg/m²) = | (20 mg/m²) | (1.5 mg) |
| | Approximately 15 minute IV treatment | 30 minute IV | |
| | Cyclophosphamide (500 mg/m²) = | infusion | |
| | Approximately 30 minute IV treatment | | |
| | Docetaxel (75 mg/m²) = | | |
| | Approximately 60 minute IV treatment | | |

Abbreviations: IV = intravenous; SC = subcutaneous
Note:
During Cycles 2 to 4, the doxorubicin component may be omitted at the discretion of the investigator, i.e., TC will be administered instead of TAC.

Patients have been randomized to receive TAC (or TC for Cycles 2 to 4)+pegfilgrastim or a combination of plinabulin and pegfilgrastim in each of Arms 1, 2, 3, and 4.

In Phase 2 (Open Label), Cycles 1 to 4 consisted of TAC (or TC for Cycles 2 to 4) administered IV on Day 1, every 21 days. Patients in Arms 2 and 3 received a single dose of plinabulin over 30 minutes (±5 minutes), 30 minutes after the end of the TAC (or TC for Cycles 2 to 4) infusion on Day 1. On Day 2 of each cycle (≥24 hours after completing chemotherapy) patients in Arm 1 received a single dose of pegfilgrastim (6.0 mg) (subcutaneous injection) in an open label treatment.

Docetaxel: Docetaxel was administered at a dose of 75 mg/m2. Administration was carried out with a 1-hour IV infusion per institutional protocol at the dose prescribed by this clinical study protocol (75 mg/m2). Dexamethasone (16 mg per day administered as 8 mg twice daily, or as per institution standard) was given on the day before, the day of (Day 1), and the day following docetaxel infusion (Day 2).

Doxorubicin: Doxorubicin was administered at a dose of 50 mg/m2. Doxorubicin is potentially cardiotoxic. Risk for doxorubicin cardiotoxicity increases with the cumulative lifetime dose of doxorubicin. At the doxorubicin dose and schedule in this study, patients received a cumulative doxorubicin dose of 240 mg/m2 of body surface area, below the threshold for symptomatic cardiac dysfunction. Patients were monitored, per institutional standard, for doxorubicin cardiotoxicity.

During Cycles 2 to 4, the doxorubicin component may be omitted at the discretion of the investigator, i.e., TC can be administered instead of TAC.

Cyclophosphamide: Cyclophosphamide was administered at a dose of 500 mg/m².

TAC Regimen: All patients received 3 week cycles of TAC chemotherapy. In each cycle, doxorubicin (50 mg/m2) given as a 15-minute IV infusion was administered first, followed immediately by cyclophosphamide (500 mg/m2) given as a 30-minute IV infusion, and then by docetaxel (75 mg/m2) administered as 1-hour IV infusion (the infusion times stated are approximate). Patients receiving TAC chemotherapy as an adjuvant treatment for their early breast cancer, received 4 cycles of TAC chemotherapy and at the discretion of the investigator up to 6 cycles of TAC chemotherapy (i.e., after completion of the 4 cycles on the protocol, these patients continue to receive TAC chemotherapy but with open label pegfilgrastim to prevent neutropenia).

TAC has a high risk (>20%) of causing FN. The NCCN guidelines recommend routine, primary prophylaxis with myeloid growth factor support in the treatment patients with high risk regimens such as TAC. During Cycles 2 to 4, the doxorubicin component may be omitted at the discretion of the investigator, i.e., TC may be administered instead of TAC.

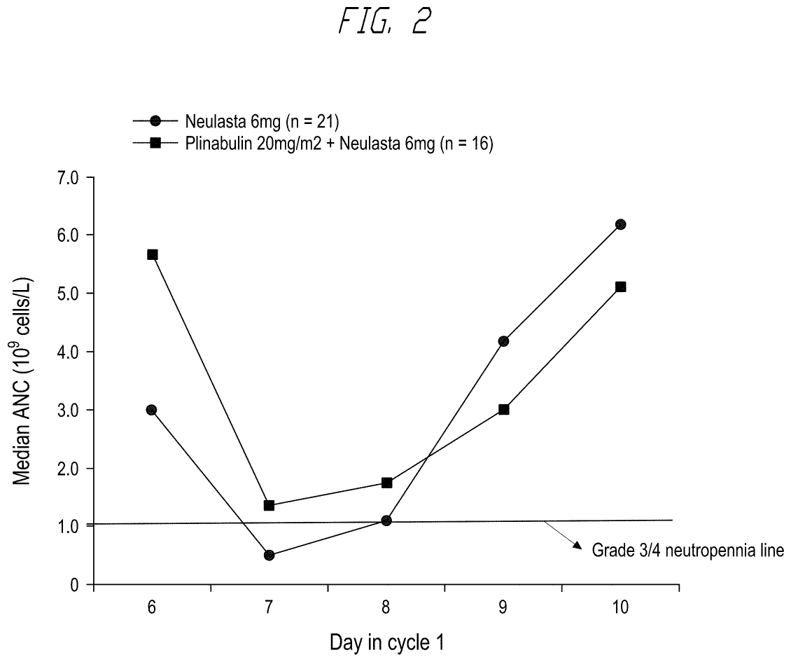

The combination of plinabulin and G-CSF (e.g., pegfilgrastim) were surprisingly effective in reducing neutropenia. FIG. 2 shows the median ANC in the pegfilgrastim group and the plinabulin (20 mg/m2) and pegfilgrastim (6 mg) group. In FIG. 2, the plinabulin (20 mg/m2) and pegfilgrastim (6 mg) combination kept the ANC nadir within normal range, while the pegfilgrastim group showed the ANC nadir to be within the grade 3/4 neutropenia range.

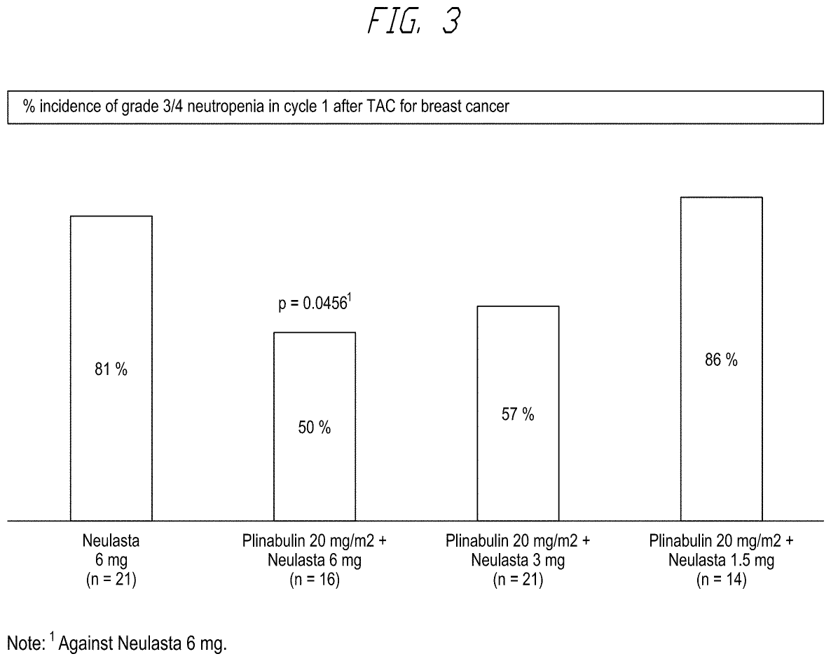

The study results also showed that the plinabulin and pegfilgrastim combination significantly reduced the incidence of severe neutropenia. In FIG. 3, the group with pegfilgrastim (6 mg) showed 81% incidence of developing grade 3/4 neutropenia in cycle 1 after TAC treatment for breast cancer, while the group with plinabulin (20 mg/m²) and pegfilgrastim (6 mg) showed 50% incidence of developing grade 3/4 neutropenia, and the group with plinabulin (20 mg/m²) and pegfilgrastim (3 mg) showed 57% incidence of developing grade 3/4 neutropenia in cycle 1 after TAC treatment for breast cancer.

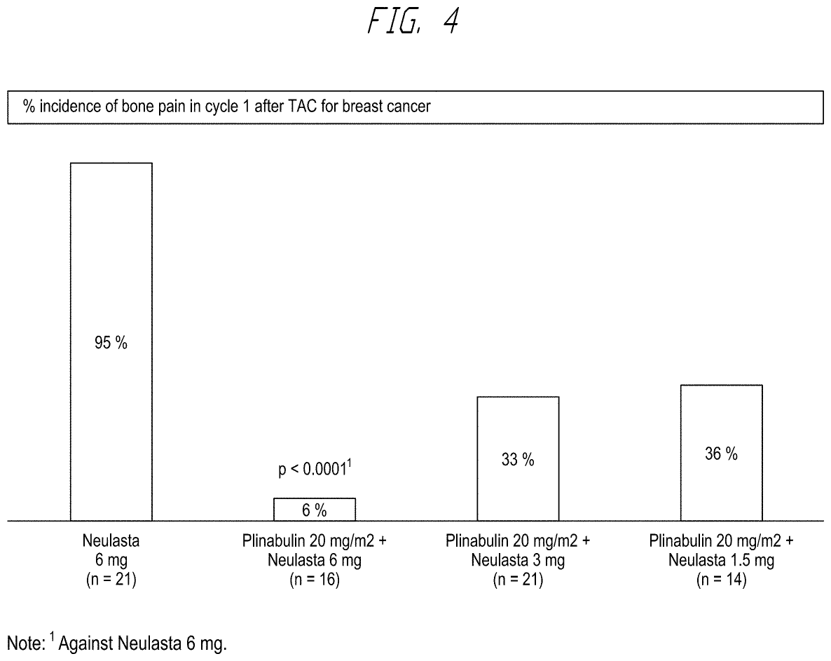

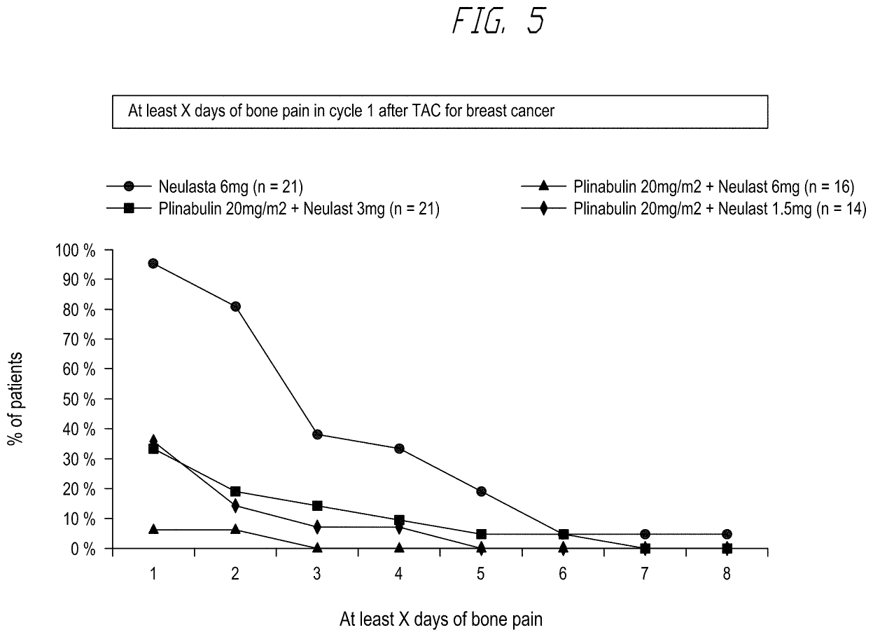

In addition, the plinabulin and pegfilgrastim combination significantly reduced the incidence of developing bone pain and also shortened the duration of bone pain. In FIG. 4, the group with pegfilgrastim (6 mg) showed 95% incidence of bone pain in cycle 1 after TAC treatment for breast cancer, while the group with plinabulin (20 mg/m²) and pegfilgrastim (6 mg) showed 6% incidence of bone pain, the group with plinabulin (20 mg/m²) and pegfilgrastim (3 mg) showed 33% incidence of bone pain, and the group with plinabulin (20 mg/m²) and pegfilgrastim (1.5 mg) showed 36% incidence of bone pain in cycle 1 after TAC treatment for breast cancer. In FIG. 5, the group with pegfilgrastim (6 mg) showed over 90% patients having at least one day of bone pain, and over 80% patients having at least two days of bone pain, close to 40% patients having at least three days of bone pain in cycle 1 after TAC treatment for breast cancer. In comparison, the group with plinabulin (20 mg/m²) and pegfilgrastim (6 mg) showed less than 10% patients having at least one day of bone pain and no patients reporting having at least three days of bone pain. The group with plinabulin (20 mg/m²) and pegfilgrastim (3 mg) showed less than 40% of patients having at least one day of bone pain, and the group with plinabulin (20 mg/m²) and pegfilgrastim (1.5 mg) also showed less than 40% patients having bone pain in cycle 1 after TAC treatment for breast cancer.

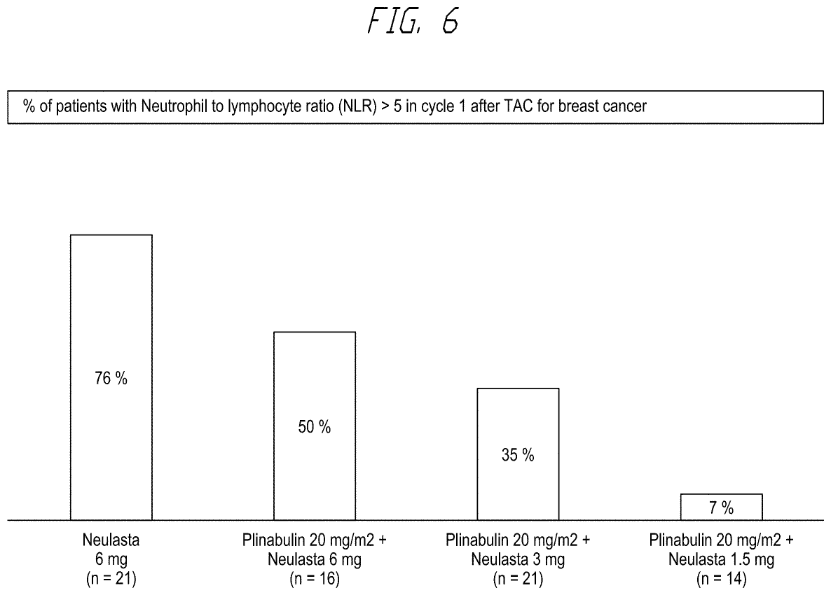

The study results also showed that plinabulin was effective to alleviate the immune suppression effect of pegfilgrastim as measured using the neutrophil to lymphocyte ratio (NLR) and LMR (lymphocyte to monocyte ratio). FIG. 6 shows the percentage of patients having a NLR value of greater than 5 in the various treatment groups. An elevated NLR value is often associated with immune suppression and a poor prognosis in cancer treatment. In FIG. 6, the group with pegfilgrastim (6 mg) showed 76% of patients with NRL greater than 5 in cycle 1 after TAC treatment for breast cancer, while the group with plinabulin (20 mg/m²) and pegfilgrastim (6 mg) showed 50% of patients with NRL greater than 5, the group with plinabulin (20 mg/m²) and pegfilgrastim (3 mg) showed 35% of patients with NRL greater than 5, and the group with plinabulin (20 mg/m²) and pegfilgrastim (1.5 mg) showed 7% of patients with NRL greater than 5 in cycle 1 after TAC treatment for breast cancer.

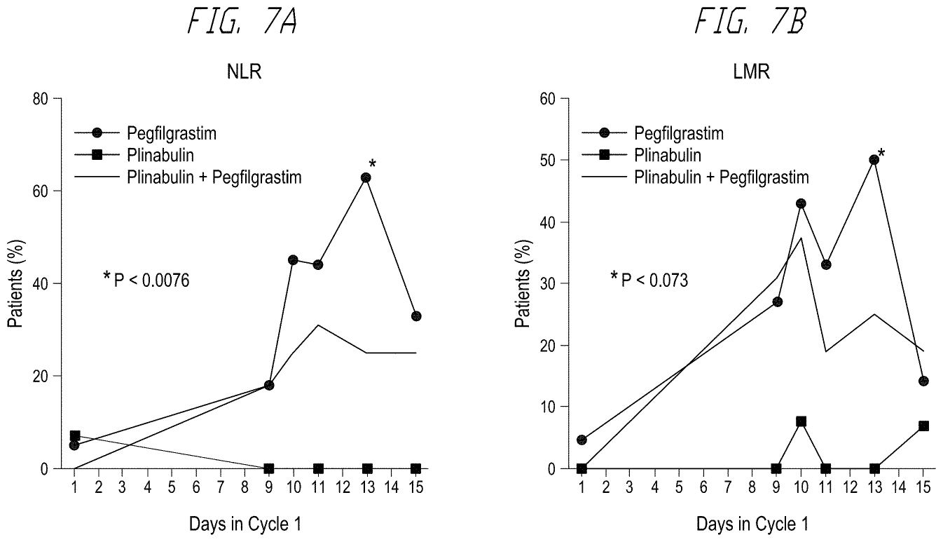

FIG. 7A shows the percentage of patients with NLR over 5 in plinabulin (20 mg/m2) alone, plinabulin (20 mg/m2)+neulasta 6 mg, and Neulasta (6 mg). FIG. 7B shows the percentage of patients with LMR less than 3.2 in plinabulin (20 mg/m2) alone, plinabulin (20 mg/m2)+neulasta 6 mg, and Neulasta (6 mg).

TABLE 3
summarizes the study results in cycle 1 after TAC treatment for breast cancer

| | | Plinabulin | Plinabulin | Plinabulin |
|---|---|---|---|---|
| | | 20 mg/m² + | 20 mg/m² + | 20 mg/m² + |
| | pegfilgrastim | pegfilgrastim | pegfilgrastim | pegfilgrastim |
| | 6 mg (n = 21) | 6 mg (n = 16) | 3 mg (n = 21) | 1.5 mg (n = 14) |
| DSN (grade 3/4) | 1.4 ± 1.0 | 0.9 ± 1.1 | 1.6 ± 1.6 | 2.6 ± 1.6 |
| | | (36% decrease) | | |
| % neutropenia | 57% | 38% | 52% | 57% |
| (grade 4) | | (33% decrease) | | |
| % neutropenia | 81% | 50% | 57% | 86% |
| (grade 3/4) | | | | |
| % bone pain (at | 95% | 6% | 33% | 36% |
| least one day) | | | | |

TABLE 4
Comparison of the plinabulin and pegfilgrastim (6
mg) combination and pegfilgrastim standard of care

| | | Plinabulin |
|---|---|---|
| | | 20 mg/m² + |
| | | pegfilgrastim |
| | pegfilgrastim 6 mg | 6 mg |
| DSN (grade 3/4) | Well over 1 day | Less than 1 day |
| % neutropenia | High (>80%) | Low (50%) |
| (grade 3/4) | | |
| Median ANC Nadir | 0.47 (>50% grade 4 | 1.00 (>50% |
| (10\hat{ }9 cells/L) | neutropenia) | avoiding grade |
| | | 3/4 neutropenia) |
| % bone pain | Almost all | Limited |
| Anti-cancer | No | Yes |

Table 4 shows the superior profile of the plinabulin and pegfilgrastim combination versus the pegfilgrastim treatment.

### Example 3

A multicenter, randomized study, with Phase 3 is performed. The phase 3 portion is double blind. An estimated total of 180 patients with breast cancer can be enrolled in Phase 3 part of this study. Patients are stratified by region (China and Japan vs rest of the world).

Patients receive up to 4 cycles of a docetaxel/doxorubicin/cyclophosphamide based chemotherapy regimen, every 3 weeks (21 days). On Day 1 of Cycle 1, all patients receive docetaxel (75 mg/m²), doxorubicin (50 mg/m²), and cyclophosphamide (500 mg/m²)-Taxotere, Adriamycin and cyclophosphamide (TAC).

In Phase 3 (double blinded treatment), Cycles 1 to 4 consist of TAC (or TC for Cycles 2 to 4) administered IV on Day 1, every 21 days. Patients receive a single dose of plinabulin or placebo IV over 30 minutes (±5 minutes) in a double blinded manner, 30 minutes after the end of the TAC (or TC for Cycles 2 to 4) infusion. On Day 2 of each cycle (≥24 hours after completing chemotherapy) patients receive a single dose of pegfilgrastim (6.0 mg) or placebo (subcutaneous injection) in a double blinded manner. Plinabulin and the matching placebo are administered in an equal volume. Pegfilgrastim is administered at a dose of 6 mg as a single dose syringe. The matching placebo is administered in an equal volume.

### Example 4

The combination of G-CSF (e.g., pegfilgrastim or filgrastim) and plinabulin is tested for its effect in reducing neutropenia induced by chemotherapy or radiation therapy. Patients having cancer receiving myelosuppresive chemotherapy or radiation therapy are assigned into the following groups: Arm (1) administration of a combination of G-CSF (e.g., pegfilgrastim or filgrastim) in the range of about 1 mg-25 mg (e.g., 0.1 mg-6 mg, 1 mg-5.5 mg, 2 mg-5.5 mg, 2 mg-4 mg, 3 mg-6 mg, 3 mg-5.5 mg, 4 mg-5.5 mg, or less than 6 mg) and plinabulin in the range of about 1 mg/m²-50 mg/m² (e.g., 1-20, 1-30, 5-10, 5-30, 5, 10, 20, 30 mg/m²); Arm (2) administration of G-CSF (e.g., pegfilgrastim or filgrastim) alone; Arm (3) administration of plinabulin alone; and Arm (4) administration of placebo.

Plinabulin or Matching Placebo: Plinabulin is administered at a selected dose ((e.g., 1-20, 1-30, 5-10, 5-30, 5, 10, 20, 30 mg/m²). In one control group, matching placebo is administered in an equal volume. Pegfilgrastim or Matching Placebo: Pegfilgrastim is administered subcutaneously at the selected dose (e.g., 0.1 mg-6 mg, 1 mg-5.5 mg, 2 mg-5.5 mg, 2 mg-4 mg, 3 mg-6 mg, 3 mg-5.5 mg, 4 mg-5.5 mg, or less than 6 mg) as a single dose syringe. In another control group, matching placebo is administered in an equal volume.

The population pharmacokinetics approach can be used to characterize the pharmacokinetics of plinabulin and pegfilgrastim following the administration of a chemotherapy or radiotherapy. The pharmacodynamics assessments include blood pressure and DSN in various cycles of the study.

It is expected that the combination of G-CSF (e.g., pegfilgrastim or filgrastim) and plinabulin is effective in reducing incidence of neutropenia, particularly severe grade 3/4 neutropenia and the combination can maintain the patient's neutrophil count to allow for continued chemotherapy treatment.

What is claimed is:

1. A method of treating a chemotherapy induced neutropenia, comprising co-administering plinabulin and one or more G-CSF drugs.

2. A method of stimulating neutrophil survival, comprising co-administering plinabulin and one or more G-CSF drugs.

3. The method of claim 1 or 2, wherein the G-CSF drug is pegfilgrastim.

4. The method of claim 1 or 2, wherein the G-CSF drug is selected from Neupogen®, Tevagrastim®, Biograstim®, Ratiograstim®, Zarxio®, Filgrastim Hexal®, Neulasta®, Granocyte®, Neutrogin®, Neu-up®, Rolontis®, Aiduo (mecapegfilgrastim, Hengrui), and Fulphila®.

5. The method of any one of claims 1-4, wherein the total dosage of the G-CSF drug used in a 21-day cycle is in the range of about 0.1 mg to about 20 mg.

6. The method of claim 5, wherein the total dosage of the G-CSF drug used in a 21-day cycle is less than about 6 mg.

7. The method of claim 5, wherein the total dosage of the G-CSF drug used in a 21-day cycle is about 1.5 mg.

8. The method of claim 5, wherein the total dosage of the G-CSF drug in a 21-day cycle is about 3 mg.

9. The method of claim 5, wherein the total dosage of the G-CSF drug in a 21-day cycle is about 6 mg.

10. The method of any one of claims 1-9, wherein the G-CSF drug is administered in a single dose in a 21-day cycle.

11. The method of any one of claims 1-9, wherein the G-CSF drug is administered in two or more doses in a 21-day cycle.

12. The method of any one of claims 1-11, wherein the G-CSF drug is administered using an on-body injector.

13. The method of any one of claims 1-12, wherein the G-CSF drug is administered subcutaneously.

14. The method of any one of claims 1-13, comprising administering the G-CSF drug at least 24 hours after the administration of the chemotherapy.

15. The method of any one of claims 1-13, comprising administering the G-CSF drug within 24 hours after the administration of the chemotherapy.

16. The method of any one of claims 1-13, comprising administering the G-CSF drug when the patient has an absolute neutrophil count that is lower than about 1.5×10⁹/L.

17. The method of any one of claims 1-13, comprising administering two or more doses of the G-CSF drug wherein a first dose of the G-CSF drug is administered between about 24 hours and 48 hours after the administration of the chemotherapy.

18. The method of any one of claims 1-17, comprising administering plinabulin within 24 hours after the administration of the chemotherapy.

19. The method of any one of claims 1-18, comprising administering plinabulin within 2 hours after the administration of the chemotherapy.

20. The method of any one of claims 1-19, comprising administering plinabulin within 1 hour after the administration of the chemotherapy.

21. The method of any one of claims 1-20, comprising administering a single dose of plinabulin in a 21-day treatment cycle.

22. The method of any one of claims 1-21, comprising administering plinabulin at a total dosage in the range of about 1 mg/m² to about 50 mg/m² in a 21-day cycle.

23. The method of any one of claims 1-22, comprising administering plinabulin at a total dosage less than or equal to 40 mg/m² in a 21-day cycle.

24. The method of any one of claims 1-23, comprising administering plinabulin at a total dosage of about 10 mg/m² in a 21-day cycle.

25. The method of any one of claims 1-23, comprising administering plinabulin at a total dosage of about 20 mg/m² in a 21-day cycle.

26. The method of any one of claims 1-23, comprising administering plinabulin at a total dosage of about 30 mg/m² in a 21-day cycle.

27. The method of any one of claims 1-23, comprising administering plinabulin at a total amount in the range of about 10 mg to about 60 mg in a 21-day cycle.

28. The method of any one of claims 1-23, comprising administering plinabulin at a total amount of about 40 mg in a 21-day cycle.

29. The method of any one of claims 1-27, wherein the chemotherapy comprises administering docetaxel and no other chemotherapeutic agent.

30. The method of any one of claims 1-27, wherein the chemotherapy comprises administering docetaxel, doxorubicin and cyclophosphamide (TAC); docetaxel and cyclophosphamide (TC); doxorubicin and cyclophosphamide (AC); docetaxel and doxorubicin (TA); docetaxel; doxorubicin; or cyclophosphamide.

31. The method of any one of claims 1-27, wherein the chemotherapy does not comprise docetaxel.

32. The method of any one of claims 1-31, wherein the patient has an advanced or metastatic breast cancer, early breast cancer, non-small cell lung cancer, refractory metastatic prostate cancer.

33. The method of any one of claims 1-31, wherein the patient has head and neck cancer, lung cancer, stomach cancer, colon cancer, pancreatic cancer, prostate cancer, breast cancer, kidney cancer, bladder cancer, ovary cancer, cervical cancer, melanoma, glioblastoma, myeloid leukemia, myeloma, lymphoma, or leukemia.

34. The method of any one of claims 1-33, wherein the plinabulin is administered less than 1 hour after the administration of the chemotherapy.

35. The method of any one of claims 1-34, wherein the plinabulin is administered about 30 mins after the administration of the chemotherapy.

36. The method of any one of claims 1-35, wherein the neutropenia is a grade 3 or 4 neutropenia.

37. The method of any one of claims 1-36, wherein the neutropenia is a grade 4 neutropenia.

38. The method of any one of claims 1-37, comprising reducing an incidence of grade 3 or 4 neutropenia by at least 5%.

39. The method of any one of claims 1-38, comprising reducing a duration of grade 3 or 4 neutropenia by at least about 2 times.

40. A kit comprising a chemotherapeutic agent, about 1 mg to about 80 mg of plinabulin, and about 0.1 mg to about 20 mg of G-CSF, wherein the chemotherapy, G-CSF, and the plinabulin are provided in separate sterile containers.

41. The kit of claim 40, wherein the amount of plinabulin is less than 50 mg.

42. The kit of claim 40, wherein the amount of plinabulin is about 10 mg.

43. The kit of claim 40, wherein the amount of plinabulin is about 20 mg.

44. The kit of claim 40, wherein the amount of plinabulin is about 30 mg.

45. The kit of claim 40, wherein the amount of plinabulin is about 40 mg.

46. The kit container of any one of claims 40-45, wherein the amount of G-CSF is less than 10 mg.

47. The kit of any one of claims 40-46, wherein the amount of G-CSF is about 6 mg.

48. The kit of any one of claims 40-46, wherein the amount of G-CSF is about 3 mg.

49. The kit of any one of claims 40-46, wherein the amount of G-CSF is about 1.5 mg.

50. A method of reducing bone pain induced by a G-CSF drug, comprising administering an effective amount of plinabulin.

51. A method of alleviating immune suppression effect induced by a G-CSF drug, comprising administering an effective amount of plinabulin.

52. A method of treating a chemotherapy induced neutropenia or stimulating neutrophil survival, comprising one or more cycles of a chemotherapy regimen, each cycle of the chemotherapy regimen independently comprising:
administering one or more chemotherapeutic agents on day 1,
administering plinabulin on day 1, and
administering one or more G-CSF drugs on day 2,
wherein the plinabulin is administered within 12 hours, within 4 hours, within 2 hours, or within 1 hour after the administration of the one or more chemotherapeutic agents.

53. The method of claim 52, wherein the one or more G-CSF drugs are administered within 24 hours after the administration of the plinabulin.

54. The method of claim 52, comprising two to four cycles of the chemotherapy regimen.

55. The method of claim 52, wherein each cycle of the chemotherapy regimen independently lasts up to 30 days.

56. The method of claim 55, wherein each cycle of the chemotherapy regimen lasts 21 days.

57. The method of claim 52, wherein the one or more chemotherapeutic agents are selected, independently in each cycle of the chemotherapy regimen, from the group consisting of: docetaxel, doxorubicin, and cyclophosphamide.

58. The method of claim 56, wherein the plinabulin is administered, independently in each cycle of the chemotherapy regimen, in a single or multiple doses of up to about 40 mg/m² about 10 to 50 minutes, or about 20 to 40 minutes after the administration of the one or more chemotherapeutic agents.

59. The method of claim 56, wherein the plinabulin is administered, independently in each cycle of the chemotherapy regimen, in a single dose of about 5 mg/m², about 10 mg/m², or about 20 mg/m² about 25 to 35 minutes, or about 28 to 32 minutes, or about 30 minutes after the administration of the one or more chemotherapeutic agents.

60. The method of claim 52, wherein the one or more G-CSF drugs are selected, independently in each cycle of the chemotherapy regimen, from the group consisting of: Neupogen®, Tevagrastim®, Biograstim®, Ratiograstim®, Zarxio®, Filgrastim Hexal®, Neulasta®, Granocyte®, Neutrogin®, Neu-up®, Rolontis®, Aiduo (mecapegfilgrastim, Hengrui), and Fulphila®.

61. The method of claim 52, wherein the one or more G-CSF drugs is pegfilgrastim.

62. The method of claim 52, wherein the one or more G-CSF drugs are administered, independently in each cycle of the chemotherapy regimen, in a single or multiple doses of up to about 6 mg at least about 24 hours after the administration of the one or more chemotherapeutic agents.

63. The method of claim 52, wherein the one or more G-CSF drugs are administered, independently in each cycle of the chemotherapy regimen, in a single or multiple doses of up to about 6 mg about 20 to about 50 hours after the administration of plinabulin.

64. The method of claim 52, wherein the one or more G-CSF drugs are administered, independently in each cycle of the chemotherapy regimen, in a single dose of about 1.5 mg, about 3.0 mg, or about 6 mg about 20 to about 30 hours after the administration of plinabulin.

65. Plinabulin for use in combination with one or more G-CSF drugs for treating a chemotherapy induced neutropenia.

66. Plinabulin for use in combination with one or more G-CSF drugs for stimulating neutrophil survival.

67. Plinaublin for use in reducing bone pain induced by a G-CSF drug.

68. Plinaublin for use in alleviating an immune suppression effect induced by a G-CSF drug.

69. Plinabulin for use in combination with one or more G-CSF drugs for treating a chemotherapy induced neutropenia or stimulating neutrophil survival in one or more cycles of a chemotherapy regimen, each cycle of the chemotherapy regimen independently comprising:
administering one or more chemotherapeutic agents on day 1,
administering plinabulin on day 1, and
administering one or more G-CSF drugs on day 2,
wherein the plinabulin is administered within 12 hours, within 4 hours, within 2 hours, or within 1 hour after the administration of the one or more chemotherapeutic agents.
